(19) United States
(12) Patent Application Publication
(10) Pub. No.: US 2025/0295353 A1
(43) Pub. Date: Sep. 25, 2025

(54) COMPUTER-IMPLEMENTED METHODS AND SYSTEMS FOR ANALYSIS OF NEUROLOGICAL IMPAIRMENT

(71) Applicant: HOFFMANN-LA ROCHE INC., Little Falls, NJ (US)

(72) Inventors: Frank DONDELINGER, Basel (CH); Marcin ELANTKOWSKI, Basel (CH); Chenfu SHI, Basel (CH)

(21) Appl. No.: 18/869,699

(22) PCT Filed: May 24, 2023
(86) PCT No.: PCT/EP2023/063976

§ 371 Date: Nov. 26, 2024

(30) Foreign Application Priority Data
May 31, 2022 (EP) 22176582.9

(51) Int. Cl. A61B 5/00; A61B 5/11; G16H 10/60; G16H 50/20

(52) U.S. Cl. CPC A61B 5/4082; A61B 5/1124; A61B 5/4842; A61B 5/7267; A61B 5/748; G16H 10/60; G16H 50/20

(57) ABSTRACT

A computer-implemented method of generating an analytical model for tracking or predicting the progression of a neurological impairment comprises: receiving training data comprising the results of a plurality of digital tests of neurological impairment; and training the analytical model using the received training data, thereby generating the analytical model. Corresponding com-puter-implemented methods for extracting feature data from the results of a digital test of neurological impairment, and for tracking or predicting the status or process of a neurological impairment are also provided.

### TECHNICAL FIELD

The present invention relates to the field of digital assessment of diseases. In particular, the present invention relates to computer-implemented methods and systems for generating an analytical model for tracking or predicting the progression of a neurological impairment.

### BACKGROUND

Diseases, particularly neurological diseases, require intensive diagnostic measures for disease management. These diseases are typically progressive diseases and need to be evaluated by a staged system in order to determine the precise status of the disease sufferer. Prominent examples among progressive neurological diseases are multiple sclerosis (MS), Huntington's Disease (HD) and spinal muscular atrophy (SMA).

Currently, the staging of such diseases requires great effort and is cumbersome for the patients. Typically, the patients need to go to medical specialists in hospitals or doctor's offices. Moreover, staging requires experience on the part of the medical specialist and is often subjective and based on personal experience and judgement. Nevertheless, there are some parameters from disease staging which are particularly useful for disease management. Moreover, there are other cases such as in SMA were a clinically relevant parameter such as the forced vital capacity needs to be determined by special equipment, i.e. spirometric devices.

For all of these cases, it might be helpful to determine surrogates. Suitable surrogates include biomarkers and, in particular, digitally acquired biomarkers such as performance parameters from tests which am at determining performance parameters of biological functions that can be correlated to the staging systems or that can be surrogate markers for the clinical parameters.

### SUMMARY OF THE INVENTION

At a high-level, the present invention aims to improve the efficiency with which meaningful clinical outputs may be derived from the results of tests of neurological impairment. Specifically, the invention relates to the generation of an analytical model such as a machine-learning model based on training data. In some, preferred cases, an autoencoder is used, and latent variables which may be useful predictors or indicators of a neurological impairment can be identified. Accordingly, a first aspect of the present invention may provide a computer-implemented method of generating an analytical model for tracking or predicting the progression of a neurological impairment, the computer-implemented method comprising: receiving training data comprising the results of a plurality of digital tests of neurological impairment; and training the analytical model using the received training data, thereby generating the analytical model. Herein, an “analytical model” may alternatively be referred to as an “analysis model”, which is defined later in this application.

The analytical model may be a machine-learning model such as an autoencoder, or a variational autoencoder. Autoencoders and variational autoencoders may comprise an encoder. An encoder may be configured to generate, from an input data set comprising a first number of variables, an intermediate data set in the form of a latent vector comprising a second number of latent variables, the second number being less than the first number. The intermediate data set in the form of a latent vector may be referred to herein as a latent representation, the entries in the latent vector being referred to as latent variables, explained in more detail elsewhere. Accordingly, the encoder may comprise a latent distribution determination module configured to determine, for each of the latent variables, a respective latent distribution, each latent distribution being a probability distribution for the value of the latent variable corresponding to the respective dimension in latent space. Methods of generating latent distributions already exist 1. The latent distribution may be in the form of a Gaussian distribution which is defined in terms of a mean and standard deviation, and/or the latent variables all have a prior distribution, optionally wherein the prior distribution is the same for all variables and/or is a Gaussian distribution with mean equal to 0 and standard deviation equal to 1.

Training the analytical model may comprise training an encoder model to learn a latent representation of the training data, wherein a latent representation comprises a plurality of latent variables, e.g. in the form of a latent vector as outlined in the previous paragraph. Herein “latent variables” may be used to refer to variables which are not directly observed, but which are inferred through e.g. a mathematical model (such as the analytical model) from other directly-measured variables (i.e. the results of the digital test of neurological impairment). Training the analytical model may comprise training the analytical model to predict a metric indicative of neurological impairment. This may comprise using one or more latent variables of an encoder trained to learn a representation of data comprising the results of a plurality of digital tests of neurological impairment. The latent representation preferably comprises a plurality of latent variables.

Training an encoder may comprise, training the encoder as part of an analytical model to learn a latent representation of the input data and reconstruct the input data from the latent representation, wherein training minimizes reconstruction loss. In some cases, where the encoder is trained as part of an autoencoder (see later disclosure) training may not minimize divergence between a prior distribution and a latent distribution. In other cases, in which the encoder is trained as part of a variational autoencoder (again, see later disclosure), training may minimize divergence between a prior distribution and a latent distribution.

1 https://towardsdatascience.com/understanding-variational-autoencoders-vaes-f70510919f73#:˜:text=variational%20autoencoders%20(VAEs)%20are%20aut oencoders,order&20to%20ensure%20a%20better

Once the latent distribution has been determined, it may be necessary to sample the values of the latent variables according to the latent distribution. In order to achieve this, the encoder may comprise a sampling module configured to determine the value of each of the second number of latent variables by sampling the respective latent distribution associated with that latent variable.

To obtain a meaningful clinical output, the computer-implemented method may further comprise using one or more of the latent variables as predictive features of an analytical model for tracking or predicting the progression of a neurological impairment. This is in contrast to earlier computer-implemented methods in which specific, predetermined features were extracted from the results of the digital test of neurological impairment. Through the use of latent variables as proposed by the present invention, it is possible to extract useful information from previously unknown or unmeasurable variables. This enables a greater degree of flexibility, and reduced the burden of identifying specific features which correlate well with e.g. the status or progression of a neurological impairment. In some cases, using one or more of the latent variables as predictive features of an analytical model for tracking or predicting the progression of a neurological impairment may comprise training an analytical model to predict one or more metrics indicative of the status or progression of neurological impairment in a supervised manner.

The analytical model may comprise the encoder. Preferably, the encoder has been trained, or is trained in an unsupervised manner as part of an autoencoder. In the context of the present application, an autoencoder is a type of artificial neural network which may be used to discover structure within data in order to develop a compressed (latent) representation of the input. The compressed representation is preferably defined in terms of the latent variables discussed elsewhere in this application. The encoder may comprise one or more long short-term memory (LSTM) networks. The encoder may, additionally or alternatively, comprise one or more convolutional neural networks (CNNs).

In implementations of the invention in which the encoder forms part of an autoencoder, the encoder may further comprise a decoder, which may be configured to generate, from an intermediate data set comprising the second number of latent variables, an output data set comprising a third number of variables. The third number is preferably greater than the second number. Alternatively, or additionally, the machine-learning model may comprise a decoder configured to generate, from an input data set comprising the latent variables of the encoder, an output data set that reproduces (or aims to reproduce) the input data provided to the encoder. Such decoders may include one or more LSTM networks and/or one or more CNNs. In the cases where the encoder is trained as part of an autoencoder, the decoder may be trained at the same time, as part of a single end-to-end model.

In some implementations, the computer-implemented method may further comprise: at least partially retraining an encoder previously trained using different training data, and/or wherein training the encoder is performed by transfer learning. In this way, it may be possible to enhance the training of the encoder using data, such as previously obtained ground truth data, which has already been used for training, e.g. in a supervised manner, to provide an improved encoder. In the context of the present invention, the term “transfer learning” is used to refer to the application of knowledge gained (e.g. learned) while solving one problem, and applying that knowledge to a different, but related, problem.

Training the encoder model may, additionally or alternatively, comprise: training the encoder model as part of an analytical model configured to predict one or more metrics indicative of the status or progression of neurological impairment, and/or wherein training the encoder model comprises training the encoder model in a supervised manner using training data comprising the value of one or more metrics indicative of the status or progression of neurological impairment.

Training the analytical model may comprise applying the analytical model to each set of results of the training data to generate respective corresponding output data; and varying one or more operating parameters of the analytical model in order to minimize a loss function parameterizing a deviation between the output data and the respective input data. This corresponds to the minimization of reconstruction loss which was alluded to earlier in this application. In other words, the loss function may be a construction loss component indicative of the deviation between the input data and the output data. Reconstruction loss corresponds to the error in recreating the original data. In implementations involving variational autoencoders, or the like, in which a latent distribution is constructed, the loss function may also comprise an indication of the extent to which the latent distribution deviations from a prior distribution. Accordingly, the loss function may comprise a distribution divergence component indicative of the deviation between a prior distribution and the determined latent distributions for each of the second number of latent variables. The prior distribution may be a normal distribution. The distribution divergence component may be a Kulback-Leibler (KL) divergence.

We now discuss in more detail the nature of the digital test of neurological impairment. In preferred implementations, the digital test may examine a user's ability to trace over a set of points defining a shape, the accuracy of the trace correlating to e.g. the status or progression of a disease. Accordingly, the data comprising the results of the digital test of neurological impairment may comprise a plurality of coordinates, each coordinate corresponding to a location of a user's finger on the touchscreen display of an electronic device at a given time, as they attempt to trace the target shape; and/or the data comprising the results of the digital test of neurological impairment comprises results of one or more draw-a-shape tests performed by one or more users, the data comprising the results of the digital test of neurological impairment comprises the value of one or more metrics indicative of the status or progression of neurological impairment, optionally wherein the values of one or more metrics indicative of the status or progression of neurological impairment include the value of a nine-hole peg test, and/or wherein the values of one or more metrics indicative of the status or progression of neurological impairment are associated with the users from which the results of the digital test of neurological impairment were acquired.

The digital test of neurological impairment may be in the form of a distal motor test. Provision of the distal motor test may comprise causing a touchscreen display of a mobile device to display an image comprising a reference start point, a reference end portion, and an indication of a reference path to be traced between the reference start point and the reference end point. Receiving the results of the distal motor test may comprise receiving an input from the touchscreen display of the mobile device, the input indicative of a test path traced by a user attempting to trace the target path on the display of the mobile device, the test path comprising: a test start point, a test end point, and a test path traced between the test start point and the test end point. The reference end point may be the same as the reference start point, and the reference path may accordingly be a closed path. In such cases, the closed path may be a square, a circle, or a figure-of-eight. Alternatively or additionally, the reference start point may be different from the reference end pint, and accordingly the reference path may be an open path. The open path may be in the form of a straight line or a spiral.

In some cases, the neurological impairment may be multiple sclerosis, Huntington's disease, or spinal muscular atrophy.

Above, we have discussed methods of generating an analytical model. As noted, the methods may further comprise further comprising using one or more parameters of the latent distributions as features of an analytical model for tracking or predicting the progression of a neurological impairment, optionally wherein the one or more parameters include the mean of one or more latent distributions.

A second aspect of the invention may provide a computer-implemented method of extracting feature data from results of a digital test of neurological impairment, the computer-implemented method comprising: receiving data comprising the results of a digital test of neurological impairment; applying an analytical model to the data, the analytical model configured to extract and output the feature data based on the data comprising the results of the digital test of neurological impairment, wherein the analytical model is generated according to the computer-implemented method of the first aspect of the invention. It will be noted that the optional features set out in respect of the first aspect of the invention may apply equally well to the second aspect of the invention, and may therefore be combined as such. Nevertheless, we explain below some particularly important optional features.

The analytical model may be in the form of a machine-learning model. In those cases, the machine-learning model may comprise an encoder configured to generate, from an input data set comprising a first number of variables, an intermediate data set in the form of a latent vector comprising a second number of latent variables; and the second number is less than the first number. Then, the computer-implemented method may further comprise extracting the feature data from the latent vector comprising the second number of latent variables. Herein, the values of one or more of the latent variables themselves may be the feature data. Using a machine-learning model (or other analytical model) in this way enables clinicians to make inferences based on latent variables which are not directly observable, but which may still result in clinically useful results. In other cases, the values of one or more of the latent variables may be used to calculate a parameter which may comprise a value on an expanded disability status scale (EDSS) which may be indicative of a status or progression of multiple sclerosis, a forced vital capacity (FVC) value which may be indicative of a status or progress of spinal muscular atrophy, or a total motor score (TMS) which may be indicative of a status or progression of Huntington's Disease.

The details of the digital test of neurological impairment have been explained previously.

A third aspect of the invention provides a computer-implemented method of tracking or predicting the progression of a neurological impairment or other disease in a subject, the computer-implemented method comprising the steps of: extracting feature data from results of a digital test of neurological impairment performed by the subject according to the computer-implemented method of the second aspect of the invention; and determining or predicting the status or progression of the neurological impairment based on the extracted feature data. Implementations of the third aspect of the invention may comprise any of the optional features set out earlier in this application in respect of the first and second aspects of the invention. We set out some important optional features below.

Extracting feature data from results of a digital test of neurological impairment may comprise providing the results of a digital test of neurological impairment as input to an encoder model trained to learn a latent representation of training data comprising the results of a plurality of digital tests of neurological impairment. Then, determining the status or progression of the neurological impairment based on the extracted feature data may comprise comparing the value of one or more latent variables from the latent representation of the data with one or more reference values. The reference values may be values of the latent variables obtained for one or more reference results of a digital test of neurological impairment, optionally wherein the one or more reference results of a digital test of neurological impairment are results from a digital test of neurological impairment performed by the same subject at a previous time point.

The aspect of the invention set out above are computer-implemented methods. Further aspects of the invention provide computer programs (or computer program products) comprising instructions, which when executed by a processor of a computer, cause the processor to execute the steps of any or all of the computer-implemented methods of the first, second, and third aspects of the invention. Other aspects of the invention comprise computer-readable media storing such computer programs. Additional aspects of the invention provide computer systems comprising a processor configured to execute the computer-implemented method of any or all of the first, second and third aspects of the invention.

### BRIEF DESCRIPTION OF THE DRAWINGS

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the drawings:

FIG. 1 shows a schematic view of a patient performing the Draw-a-Shape test.

FIG. 2 shows an example of the recorded path obtained from a patient's attempt at the Draw-a-Shape test.

FIG. 3 shows a representation of an autoencoder.

FIG. 4 shows a representation of a variational autoencoder.

FIG. 5 shows a representation of a recurrent neural network architecture used in the prior art.

FIG. 6 shows a representation of a modified recurrent neural network architecture.

FIG. 7A shows a series of graphs of recorded path data and reconstructions of those paths by an LSTM neural network architecture.

FIG. 7B shows a series of graphs of recorded path data and reconstructions of those paths by a CNN neural network architecture

FIG. 8A shows a series of graphs representing how many latent dimensions of the bottleneck layer is used in the LSTM model.

FIG. 8B shows a series of graphs representing how many latent dimensions of the bottleneck layer is used in the CNN model.

FIG. 9A shows a series of graphs visualising the output of generated interpolated data from a LSTM model.

FIG. 9B shows a series of graphs visualising the output of generated interpolated data from a CNN model.

FIG. 10A is a t-distributed stochastic neighbour (t-SNE) representation of the latent space of an LSTM model.

FIG. 10B is a t-distributed stochastic neighbour (t-SNE) representation of the latent space of a CNN model.

FIG. 11 shows a t-distributed stochastic neighbour embedding (t-SNE) representation of the latent space of each model for 10 random subjects taken from the Consonance data set.

FIG. 12 shows a Confusion matrix showing the true subject labels vs predicted subject labels for the CNN model with KL1 on consonance data. Each row and column is an individual subject.

FIG. 13 shows a series of graphs. The left-hand column is a t-SNE of the data from the Consonance study and the right hand column is a PCA of the same.

FIGS. 14A and 14B show further t-SNE representations of the Consonance data as in FIG. 13 but the points are coloured by the handcrafted feature listed above each graph.

FIG. 15 shows a heatmap graph representing the level to which a set of handcrafted features are encoded in the dimensions of the CNN model.

FIG. 16 shows a graph representing the result of a factor analysis, which aims to identify latent variables (“factors”) that generate observed variables.

FIG. 17A shows two graphs, each of a Kernel Density Estimate (KDE) plot of the PCA of the two datasets.

FIG. 17B is a graph of a scatterplot of the t-SNE of a sample of the POC and Consonance datasets.

FIG. 18 shows a scatterplot of a t-SNE representation of a sample of the two datasets by handcrafted features.

FIG. 19 shows a schematic view of the architecture of a modified autoencoder architecture.

FIG. 20 shows a graph with points representing the level of importance of each of the handcrafted features for prediction of 9HPT times.

FIG. 21 shows a graph that represents permutation importance weights vs standard deviation of the individual latents.

FIG. 22 shows a graph with points representing the level of importance of the handcrafted features and the latent representations in an unsupervised model.

FIG. 23 shows the layer weights vs the standard deviation of the latent dimensions in relation to 9HPT reconstruction.

FIG. 24A shows a graph representing the data plotted using the top latent dimensions as X and Y dimensions, where the data points are coloured by trace celerity.

FIG. 24B shows a graph representing the data plotted using the top latent dimensions as X and Y dimensions, where the data points are coloured by 9HPT times.

FIG. 25 shows a heatmap which shows the amount of mutual information between each latent dimension and handcrafted feature.

FIG. 26 shows a graph with points representing the level of importance of the handcrafted features and the latent representations in a model training on a validation data set.

FIG. 27 shows a scatter plot of Consonance 9HPT times prediction per patient visit from fully supervised models with a blue trend line, overlayed with an idealised red line where prediction matches the true time.

FIG. 28 shows the statistical results for transfer learning experiments on Floodlight POC data.

FIG. 29 shows the statistical results for transfer learning experiments on Consonance data.

FIG. 30 displays an array of graphs each representing a selection of the data from Consonance patients with large 9HPT test time changes.

FIG. 31 shows two scatterplot graphs of real 9HPT differences vs predicted 9HPT differences in Consonance patients.

FIG. 32A shows two graphs each containing a Uniform Manifold Approximation and Projection (UMAP) of the latent space of the CNN model, the upper UMAP coloured by trace celerity, and the lower UMAP coloured by 9-HPT times. Red generally represents smaller values and blue represents larger values

FIGS. 32B and 32C each show a series of graphs for the same UMAP as FIG. 32A, each coloured for a different handcrafted feature.

FIG. 33 shows a series of graphs each containing the UMAP of the latent space of the CNN model, wherein each graph highlights the data points for a single patient.

FIGS. 34A and 34B show a series of graphs each containing the UMAP using the handcrafted features.

FIG. 35 shows a series of graphs each containing the same UMAP as FIGS. 34A and 34B, wherein the results from individual patients are highlighted, with results coloured by test time.

FIG. 36 shows highly schematically an embodiment of a machine learning system for generating an analytical model for tracking or predicting the progression of a neurological impairment.

### DETAILED DESCRIPTION

There are various abbreviations used throughout the detailed description, which are defined below.
MS Multiple Sclerosis
PPMS Primary Progressive Multiple Sclerosis
HD Huntington's Disease
SMA Spinal Muscular Atrophy
AI Artificial Intelligence
CNS Central Nervous System
EDSS Expanded Disability Status Scale
kNN K Nearest Neighbours
PLS Partial Last-Squares
RF Random Forest
XT Extremely Randomized Trees
SVM Support Vector Machines
LDA Linear Discriminant Analysis
QDA Quadratic Discriminant Analysis
NB Naïve Bayes
ALU Arithmetic Logic Unit
FPU Floating-Point Unit
CPU Central Processing Unit
GPU Graphical Processing Unit
ASIC Application Specific Integrated Circuit
TPU Tensor Processing Unit
FPGA Field-Programmable Gate Array
PCA Principal Component Analysis

Aspects and embodiments of the present invention will now be discussed with reference to the accompanying figures. Further aspects and embodiments will be apparent to those skilled in the art. All documents mentioned in this text are incorporated herein by reference.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The term “machine learning” as used herein is a broad term and is to be given its ordinary and customary meaning to a person of ordinary skill in the art and is not to be limited to a special or customized meaning. The term specifically may refer, without limitation, to a method of using artificial intelligence (AI) for automatically model building of analytical models.

The term “machine learning system” as used herein is a broad term and is to be given its ordinary and customary meaning to a person of ordinary skill in the art and is not to be limited to a special or customized meaning. The term specifically may refer, without limitation, to a system comprising at least one processing unit such as a processor, microprocessor, or computer system configured for machine learning, in particular for executing a logic in a given algorithm. The machine learning system may be configured for performing and/or executing at least one machine learning algorithm, wherein the machine learning algorithm is configured for building the at least one analysis model based on the training data.

The term “analysis model” as used herein is a broad term and is to be given its ordinary and customary meaning to a person of ordinary skill in the art and is not to be limited to a special or customized meaning. The term specifically may refer, without limitation, to a mathematical model configured for predicting at least one target variable for at least one state variable. The analysis model may be a regression model or a classification model.

The term “regression model” as used herein is a broad term and is to be given its ordinary and customary meaning to a person of ordinary skill in the art and is not to be limited to a special or customized meaning. The term specifically may refer, without limitation, to an analysis model comprising at least one supervised learning algorithm having as output a numerical value within a range.

The term “classification model” as used herein is a broad term and is to be given its ordinary and customary meaning to a person of ordinary skill in the art and is not to be limited to a special or customized meaning. The term specifically may refer, without limitation, to an analysis model comprising at least one supervised learning algorithm having as output a classifier such as “ill” or “healthy”.

The term “target variable” as used herein is a broad term and is to be given its ordinary and customary meaning to a person of ordinary skill in the art and is not to be limited to a special or customized meaning. The term specifically may refer, without limitation, to a clinical value which is to be predicted. The target variable value which is to be predicted may be dependent on the disease whose presence or status is to be predicted. The target variable may be either numerical or categorical. For example, the target variable may be categorical and may be “positive” in case of presence of disease or “negative” in case of absence of the disease.

The target variable may be numerical such as at least one value and/or scale value.

For example, the disease whose status is to be predicted is multiple sclerosis. The term “multiple sclerosis (MS)” as used herein relates to disease of the central nervous system (CNS) that typically causes prolonged and severe disability in a subject suffering therefrom. There are four standardized subtype definitions of MS which are also encompassed by the term as used in accordance with the present invention: relapsing-remitting, secondary progressive, primary progressive and progressive relapsing. The term relapsing forms of MS is also used and encompasses relapsing-remitting and secondary progressive MS with superimposed relapses. The relapsing-remitting subtype is characterized by unpredictable relapses followed by periods of months to years of remission with no new signs of clinical disease activity. Deficits suffered during attacks (active status) may either resolve or leave sequelae. This describes the initial course of 85 to 90% of subjects suffering from MS. Secondary progressive MS describes those with initial relapsing-remitting MS, who then begin to have progressive neurological decline between acute attacks without any definite periods of remission. Occasional relapses and minor remissions may appear. The median time between disease onset and conversion from relapsing remitting to secondary progressive MS is about 19 years. The primary progressive subtype describes about 10 to 15% of subjects who never have remission after their initial MS symptoms. It is characterized by progressive of disability from onset, with no, or only occasional and minor, remissions and improvements. The age of onset for the primary progressive subtype is later than other subtypes. Progressive relapsing MS describes those subjects who, from onset, have a steady neurological decline but also suffer clear superimposed attacks. It is now accepted that this latter progressive relapsing phenotype is a variant of primary progressive MS (PPMS) and diagnosis of PPMS according to McDonald 2010 criteria includes the progressive relapsing variant.

Symptoms associated with MS include changes in sensation (hypoesthesia and par-aesthesia), muscle weakness, muscle spasms, difficulty in moving, difficulties with co-ordination and balance (ataxia), problems in speech (dysarthria) or swallowing (dysphagia), visual problems (nystagmus, optic neuritis and reduced visual acuity, or diplopia), fatigue, acute or chronic pain, bladder, sexual and bowel difficulties. Cognitive impairment of varying degrees as well as emotional symptoms of depression or unstable mood are also frequent symptoms. The main clinical measure of disability progression and symptom severity is the Expanded Disability Status Scale (EDSS). Further symptoms of MS are well known in the art and are described in the standard textbooks of medicine and neurology.

The term “progressing MS” as used herein refers to a condition, where the disease and/or one or more of its symptoms get worse over time. Typically, the progression is accompanied by the appearance of active statuses. The said progression may occur in all subtypes of the disease. However, typically “progressing MS” shall be determined in accordance with the present invention in subjects suffering from relapsing-remitting MS.

Determining status of multiple sclerosis, generally comprises assessing at least one symptom associated with multiple sclerosis selected from a group consisting of: impaired fine motor abilities, pins and needles, numbness in the fingers, fatigue and changes to diurnal rhythms, gait problems and walking difficulty, cognitive impairment including problems with processing speed. Disability in multiple sclerosis may be quantified according to the expanded disability status scale (EDSS) as described in Kurtzke JF, “Rating neurologic impairment in multiple sclerosis: an expanded disability status scale (EDSS)”, November 1983, Neurology. 33 (11): 1444-52. doi: 10.1212/WNL.33.11.1444. PMID 6685237. The target variable may be an EDSS value.

The term “expanded disability status scale (EDSS)” as used herein, thus, refers to a score based on quantitative assessment of the disabilities in subjects suffering from MS (Krutzke 1983). The EDSS is based on a neurological examination by a clinician. The EDSS quantifies disability in eight functional systems by assigning a Functional System Score (FSS) in each of these functional systems. The functional systems are the pyramidal system, the cerebellar system, the brainstem system, the sensory system, the bowel and bladder system, the visual system, the cerebral system and other (remaining) systems. EDSS steps 1.0 to 4.5 refer to subjects suffering from MS who are fully ambulatory, EDSS steps 5.0 to 9.5 characterize those with impairment to ambulation.

The clinical meaning of each possible result is the following:
0.0 Normal Neurological Exam
1.0 No disability, minimal signs in 1 FS
1.5 No disability, minimal signs in more than 1 FS
2.0 Minimal disability in 1 FS
2.5 Mild disability in 1 or Minimal disability in 2 FS
3.0 Moderate disability in 1 FS or mild disability in 3-4 FS, though fully ambulatory
3.5 Fully ambulatory but with moderate disability in 1 FS and mild disability in 1 or 2 FS; or moderate disability in 2 FS; or mild disability in 5 FS
4.0 Fully ambulatory without aid, up and about 12 hrs a day despite relatively severe disability. Able to walk without aid 500 meters
4.5 Fully ambulatory without aid, up and about much of day, able to work a full day, may otherwise have some limitations of full activity or require minimal assistance. Relatively severe disability. Able to walk without aid 300 meters
5.0 Ambulatory without aid for about 200 meters. Disability impairs full daily activities
5.5 Ambulatory for 100 meters, disability precludes full daily activities
6.0 Intermittent or unilateral constant assistance (cane, crutch or brace) required to walk 100 meters with or without resting
6.5 Constant bilateral support (cane, crutch or braces) required to walk 20 meters without resting
7.0 Unable to walk beyond 5 meters even with aid, essentially restricted to wheelchair, wheels self, transfers alone; active in wheelchair about 12 hours a day
7.5 Unable to take more than a few steps, restricted to wheelchair, may need aid to transfer; wheels self, but may require motorized chair for full day's activities
8.0 Essentially restricted to bed, chair, or wheelchair, but may be out of bed much of day; retains self-care functions, generally effective use of arms
8.5 Essentially restricted to bed much of day, some effective use of arms, retains some self-care functions
9.0 Helpless bed patient, can communicate and eat
9.5 Unable to communicate effectively or eat/swallow
10.0 Death due to MS

For example, the disease whose status is to be predicted is spinal muscular atrophy.

The term “spinal muscular atrophy (SMA)” as used herein relates to a neuromuscular disease which is characterized by the loss of motor neuron function, typically, in the spinal chord. As a consequence of the loss of motor neuron function, typically, muscle atrophy occurs resulting in an early dead of the affected subjects. The disease is caused by an inherited genetic defect in the SMN1 gene. The SMN protein encoded by said gene is required for motor neuron survival. The disease is inherited in an autosomal recessive manner.

Symptoms associated with SMA include areflexia, in particular, of the extremities, muscle weakness and poor muscle tone, difficulties in completing developmental phases in childhood, as a consequence of weakness of respiratory muscles, breathing problems occurs as well as secretion accumulation in the lung, as well as difficulties in sucking, swallowing and feeding/eating. Four different types of SMA are known.

The infantile SMA or SMA1 (Werdnig-Hoffmann disease) is a severe form that manifests in the first months of life, usually with a quick and unexpected onset (“floppy baby syndrome”). A rapid motor neuron death causes inefficiency of the major body organs, in particular, of the respiratory system, and pneumonia-induced respiratory failure is the most frequent cause of death. Unless placed on mechanical ventilation, babies diagnosed with SMA1 do not generally live past two years of age, with death occurring as early as within weeks in the most severe cases, sometimes termed SMA0. With proper respiratory support, those with milder SMA1 phenotypes accounting for around 10% of SMA1 cases are known to live into adolescence and adulthood.

The intermediate SMA or SMA2 (Dubowitz disease) affects children who are never able to stand and walk but who are able to maintain a sitting position at least some time in their life. The onset of weakness is usually noticed some time between 6 and 18 months. The progress is known to vary. Some people gradually grow weaker over time while others through careful maintenance avoid any progression. Scoliosis may be present in these children, and correction with a brace may help improve respiration. Muscles are weakened, and the respiratory system is a major concern. Life expectancy is somewhat reduced but most people with SMA2 live well into adulthood.

The juvenile SMA or SMA3 (Kugelberg-Welander disease) manifests, typically, after 12 months of age and describes people with SMA3 who are able to walk without support at some time, although many later lose this ability. Respiratory involvement is less noticeable, and life expectancy is normal or near normal.

The adult SMA or SMA4 manifests, usually, after the third decade of life with gradual weakening of muscles that affects proximal muscles of the extremities frequently requiring the person to use a wheelchair for mobility. Other complications are rare, and life expectancy is unaffected.

Typically, SMA in accordance with the present invention is SMA1 (Werdnig-Hoffmann disease), SMA2 (Dubowitz disease), SMA3 (Kugelberg-Welander diseases) or SMA4

SMA is typically diagnosed by the presence of the hypotonia and the absence of reflexes. Both can be measured by standard techniques by the clinician in a hospital including electromyography. Sometimes, serum creatine kinase may be increased as a biochemical parameter. Moreover, genetic testing is also possible, in particular, as prenatal diagnostics or carrier screening. Moreover, a critical parameter in SMA management is the function of the respiratory system. The function of the respiratory system can be, typically, determined by measuring the forced vital capacity of the subject which will be indicative for the degree of impairment of the respiratory system as a consequence of SMA.

The term “forced vital capacity (FVC)” as used herein refers to is the volume in liters of air that can forcibly be blown out after full inspiration by a subject. It is, typically, determined by spirometry in a hospital or at a doctor's residency using spirometric devices.

Determining status of spinal muscular atrophy, generally comprises assessing at least one symptom associated with spinal muscular atrophy selected from a group consisting of: hypotonia and muscle weakness, fatigue and changes to diurnal rhythms. A measure for status of spinal muscular atrophy may be the Forced vital capacity (FVC). The FVC may be a quantitative measure for volume of air that can forcibly be blown out after full inspiration, measured in liters, see https://en.wikipedia.org/wiki/Spirometry. The target variable may be a FVC value.

For example, the disease whose status is to be predicted is Huntington's disease.

The term “Huntington's Disease (HD)” as used herein relates to an inherited neurological disorder accompanied by neuronal cell death in the central nervous system. Most prominently, the basal ganglia are affected by cell death. There are also further areas of the brain involved such as substantia nigra, cerebral cortex, hippocampus and the purkinje cells. All regions, typically, play a role in movement and behavioral control. The disease is caused by genetic mutations in the gene encoding Huntingtin. Huntingtin is a protein involved in various cellular functions and interacts with over 100 other proteins. The mutated Huntingtin appears to be cytotoxic for certain neuronal cell types. Mutated Huntingtin is characterized by a poly glutamine region caused by a trinucleotide repeat in the Huntingtin gene. A repeat of more than 36 glutamine residues in the poly glutamine region of the protein results in the disease causing Huntingtin protein.

The symptoms of the disease most commonly become noticeable in the mid-age, but can begin at any age from infancy to the elderly. In early stages, symptoms involve subtle changes in personality, cognition, and physical skills. The physical symptoms are usually the first to be noticed, as cognitive and behavioral symptoms are generally not severe enough to be recognized on their own at said early stages. Almost everyone with HD eventually exhibits similar physical symptoms, but the onset, progression and extent of cognitive and behavioral symptoms vary significantly between individuals. The most characteristic initial physical symptoms are jerky, random, and uncontrollable movements called chorea. Chorea may be initially exhibited as general restlessness, small unintentionally initiated or uncompleted motions, lack of coordination, or slowed saccadic eye movements. These minor motor abnormalities usually precede more obvious signs of motor dysfunction by at least three years. The clear appearance of symptoms such as rigidity, writhing motions or abnormal posturing appear as the disorder progresses. These are signs that the system in the brain that is responsible for movement has been affected. Psychomotor functions become increasingly impaired, such that any action that requires muscle control is affected. Common consequences are physical instability, abnormal facial expression, and difficulties chewing, swallowing, and speaking. Consequently, eating difficulties and sleep disturbances are also accompanying the disease. Cognitive abilities are also impaired in a progressive manner. Impaired are executive functions, cognitive flexibility, abstract thinking, rule acquisition, and proper action/reaction capabilities. In more pronounced stages, memory deficits tend to appear including short-term memory deficits to long-term memory difficulties. Cognitive problems worsen over time and will ultimately turn into dementia. Psychiatric complications accompanying HD are anxiety, depression, a reduced display of emotions (blunted affect), egocentrism, aggression, and compulsive behavior, the latter of which can cause or worsen addictions, including alcoholism, gambling, and hypersexuality.

There is no cure for HD. There are supportive measurements in disease management depending on the symptoms to be addressed. Moreover, a number of drugs are used to ameliorate the disease, its progression or the symptoms accompanying it. Tetrabenazine is approved for treatment of HD, include neuroleptics and benzodiazepines are used as drugs that help to reduce chorea, amantadine or remacemide are still under investigation but have shown preliminary positive results. Hypokinesia and rigidity, especially in juvenile cases, can be treated with antiparkinsonian drugs, and myoclonic hyperkinesia can be treated with valproic acid. Ethyl-eicosapentoic acid was found to enhance the motor symptoms of patients, however, its long-term effects need to be revealed.

The disease can be diagnosed by genetic testing. Moreover, the severity of the disease can be staged according to Unified Huntington's Disease Rating Scale (UHDRS). This scale system addresses four components, i.e. the motor function, the cognition, behavior and functional abilities. The motor function assessment includes assessment of ocular pursuit, saccade initiation, saccade velocity, dysarthria, tongue protrusion, maximal dystonia, maximal chorea, retropulsion pull test, finger taps, pronate/supinate hands, luria, rigidity arms, bradykinesia body, gait, and tandem walking and can be summarized as total motor score (TMS). The motoric functions must be investigated and judged by a medical practitioner.

Determining status of Huntington's disease generally comprises assessing at least one symptom associated with Huntington's disease selected from a group consisting of: Psychomotor slowing, chorea (jerking, writhing), progressive dysarthria, rigidity and dystonia, social withdrawal, progressive cognitive impairment of processing speed, attention, planning, visual-spatial processing, learning (though intact recall), fatigue and changes to diurnal rhythms. A measure for status of is a total motor score (TMS). The target variable may be a total motor score (TMS) value. The term “total motor score (TMS)” as used herein, thus, refers to a score based on assessment of ocular pursuit, saccade initiation, saccade velocity, dysarthria, tongue protrusion, maximal dystonia, maximal chorea, retropulsion pull test, finger taps, pronate/supinate hands, luria, rigidity arms, bradykinesia body, gait, and tandem walking.

The term “Nine-Hole Peg Test (9HPT)” refers to a physiological test performed by a subject to measure finger dexterity in patients. In the test, the subject is provided with 9 pegs in a container and a pegboard with a series of 9 holes suitable for receiving the pegs such that the pegs can be readily removed again. The subject is asked to take each of the pegs and use a single hand to place them into a hole on the pegboard. Once all of the pegs are placed into board, the subject must then remove each peg one-by-one from the board and return them to the container. The subject is timed to determine how long this activity takes. This timing begins from the moment the subject touches the first peg until the final peg is returned to the container.

The term “state variable” as used herein is a broad term and is to be given its ordinary and customary meaning to a person of ordinary skill in the art and is not to be limited to a special or customized meaning. The term specifically may refer, without limitation, to an input variable which can be filled in the prediction model such as data derived by medical examination and/or self-examination by a subject. The state variable may be determined in at least one active test and/or in at least one passive monitoring. For example, the state variable may be determined in an active test such as at least one cognition test and/or at least one hand motor function test and/or or at least one mobility test.

The term “subject” as used herein, typically, relates to mammals. The subject in accordance with the present invention may, typically, suffer from or shall be suspected to suffer from a disease, i.e. it may already show some or all of the negative symptoms associated with the said disease. In an embodiment of the invention said subject is a human.

The state variable may be determined by using at least one mobile device of the subject. The term “mobile device” as used herein is a broad term and is to be given its ordinary and customary meaning to a person of ordinary skill in the art and is not to be limited to a special or customized meaning. The term may specifically refer, without limitation, to a mobile electronics device, more specifically to a mobile communication device comprising at least one processor. The mobile device may specifically be a cell phone or smartphone. The mobile device may also refer to a tablet computer or any other type of portable computer. The mobile device may comprise a data acquisition unit which may be configured for data acquisition. The mobile device may be configured for detecting and/or measuring either quantitatively or qualitatively physical parameters and transform them into electronic signals such as for further processing and/or analysis. For this purpose, the mobile device may comprise at least one sensor. It will be understood that more than one sensor can be used in the mobile device, i.e. at least two, at least three, at least four, at least five, at least six, at least seven, at least eight, at least nine or at least ten or even more different sensors. The sensor may be at least one sensor selected from the group consisting of: at least one gyroscope, at least one magnetometer, at least one accelerometer, at least one proximity sensor, at least one thermometer, at least one pedometer, at least one fingerprint detector, at least one touch sensor, at least one voice recorder, at least one light sensor, at least one pressure sensor, at least one location data detector, at least one camera, at least one GPS, and the like. The mobile device may comprise the processor and at least one database as well as software which is tangibly embedded to said device and, when running on said device, carries out a method for data acquisition. The mobile device may comprise a user interface, such as a display and/or at least one key, e.g. for performing at least one task requested in the method for data acquisition.

The term “predicting” as used herein is a broad term and is to be given its ordinary and customary meaning to a person of ordinary skill in the art and is not to be limited to a special or customized meaning. The term specifically may refer, without limitation, to determining at least one numerical or categorical value indicative of the disease status for the at least one state variable. In particular, the state variable may be filled in the analysis as input and the analysis model may be configured for performing at least one analysis on the state variable for determining the at least one numerical or categorical value indicative of the disease status. The analysis may comprise using the at least one trained algorithm.

The term “determining at least one analysis model” as used herein is a broad term and is to be given its ordinary and customary meaning to a person of ordinary skill in the art and is not to be limited to a special or customized meaning. The term specifically may refer, without limitation, to building and/or creating the analysis model.

The term “disease status” as used herein is a broad term and is to be given its ordinary and customary meaning to a person of ordinary skill in the art and is not to be limited to a special or customized meaning. The term specifically may refer, without limitation, to health condition and/or medical condition and/or disease stage. For example, the disease status may be healthy or ill and/or presence or absence of disease. For example, the disease status may be a value relating to a scale indicative of disease stage. The term “indicative of a disease status” as used herein is a broad term and is to be given its ordinary and customary meaning to a person of ordinary skill in the art and is not to be limited to a special or customized meaning. The term specifically may refer, without limitation, to information directly relating to the disease status and/or to information indirectly relating to the disease status, e.g. information which need further analysis and/or processing for deriving the disease status. For example, the target variable may be a value which need to be compared to a table and/or lookup table for determine the disease status.

The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. In particular, the communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. In particular, the communication interface may provide a data transfer connection, e.g. Bluetooth, NFC, inductive coupling or the like. As an example, the communication interface may be or may comprise at least one port comprising one or more of a network or internet port, a USB-port and a disk drive. The communication interface may be at least one web interface.

The term “input data” as used herein is a broad term and is to be given its ordinary and customary meaning to a person of ordinary skill in the art and is not to be limited to a special or customized meaning. The term specifically may refer, without limitation, to experimental data used for model building. The input data comprises the set of historical digital biomarker feature data.

The term “biomarker” as used herein is a broad term and is to be given its ordinary and customary meaning to a person of ordinary skill in the art and is not to be limited to a special or customized meaning. The term specifically may refer, without limitation, to a measurable characteristic of a biological state and/or biological condition.

The term “feature” as used herein is a broad term and is to be given its ordinary and customary meaning to a person of ordinary skill in the art and is not to be limited to a special or customized meaning. The term specifically may refer, without limitation, to a measurable property and/or characteristic of a symptom of the disease on which the prediction is based. In particular, all features from all tests may be considered and the optimal set of features for each prediction is determined. Thus, all features may be considered for each disease.

The term “digital biomarker feature data” as used herein is a broad term and is to be given its ordinary and customary meaning to a person of ordinary skill in the art and is not to be limited to a special or customized meaning. The term specifically may refer, without limitation, to experimental data determined by at least one digital device such as by a mobile device which comprises a plurality of different measurement values per subject relating to symptoms of the disease. The digital biomarker feature data may be determined by using at least one mobile device. With respect to the mobile device and determining of digital biomarker feature data with the mobile device reference is made to the description of the determination of the state variable with the mobile device above. The set of historical digital biomarker feature data comprises a plurality of measured values per subject indicative of the disease status to be predicted.

The term “historical” as used herein is a broad term and is to be given its ordinary and customary meaning to a person of ordinary skill in the art and is not to be limited to a special or customized meaning. The term specifically may refer, without limitation, to the fact that the digital biomarker feature data was determined and/or collected before model building such as during at least one test study. For example, for model building for predicting at least one target indicative of multiple sclerosis the digital biomarker feature data may be data from Floodlight POC study. For example, for model building for predicting at least one target indicative of spinal muscular atrophy the digital biomarker feature data may be data from OLEOS study. For example, for model building for predicting at least one target indicative of Huntington's disease the digital biomarker feature data may be data from HD OLE study, ISIS 44319-CS2. The input data may be determined in at least one active test and/or in at least one passive monitoring. For example, the input data may be determined in an active test using at least one mobile device such as at least one cognition test and/or at least one hand motor function test and/or or at least one mobility test.

The input data further may comprise target data. The term “target data” as used herein is a broad term and is to be given its ordinary and customary meaning to a person of ordinary skill in the art and is not to be limited to a special or customized meaning. The term specifically may refer, without limitation, to data comprising clinical values to predict, in particular one clinical value per subject. The target data may be either numerical or categorical. The clinical value may directly or indirectly refer to the status of the disease.

The processing unit may be configured for extracting features from the input data. The term “extracting features” as used herein is a broad term and is to be given its ordinary and customary meaning to a person of ordinary skill in the art and is not to be limited to a special or customized meaning. The term specifically may refer, without limitation, to at least one process of determining and/or deriving features from the input data. Specifically, the features may be pre-defined, and a subset of features may be selected from an entire set of possible features. The extracting of features may comprise one or more of data aggregation, data reduction, data transformation and the like. The processing unit may be configured for ranking the features. The term “ranking features” as used herein is a broad term and is to be given its ordinary and customary meaning to a person of ordinary skill in the art and is not to be limited to a special or customized meaning. The term specifically may refer, without limitation, to assigning a rank, in particular a weight, to each of the features depending on predefined criteria. For example, the features may be ranked with respect to their relevance, i.e. with respect to correlation with the target variable, and/or the features may be ranked with respect to redundancy, i.e. with respect to correlation between features. The processing unit may be configured for ranking the features by using a maximum-relevance-minimum-redundancy technique. This method ranks all features using a trade-off between relevance and redundancy. Specifically, the feature selection and ranking may be performed as described in Ding C., Peng H. “Minimum redundancy feature selection from microarray gene expression data”, J Bioinform Comput Biol. 2005 April; 3 (2): 185-205, PubMed PMID: 15852500. The feature selection and ranking may be performed by using a modified method compared to the method described in Ding et al., The maximum correlation coefficient may be used rather than the mean correlation coefficient and an addition transformation may be applied to it. In case of a regression model as analysis model the transformation the value of the mean correlation coefficient may be raised to the 5th power. In case of a classification model as analysis model the value of the mean correlation coefficient may be multiplied by 10.

The term “model unit” as used herein is a broad term and is to be given its ordinary and customary meaning to a person of ordinary skill in the art and is not to be limited to a special or customized meaning. The term specifically may refer, without limitation, to at least one data storage and/or storage unit configured for storing at least one machine learning model. The term “machine learning model” as used herein is a broad term and is to be given its ordinary and customary meaning to a person of ordinary skill in the art and is not to be limited to a special or customized meaning. The term specifically may refer, without limitation, to at least one trainable algorithm. The model unit may comprise a plurality of machine learning models, e.g. different machine learning models for building the regression model and machine learning models for building the classification model. For example, the analysis model may be a regression model and the algorithm of the machine learning model may be at least one algorithm selected from the group consisting of: k nearest neighbors (kNN); linear regression; partial last-squares (PLS); random forest (RF); and extremely randomized Trees (XT). For example, the analysis model may be a classification model and the algorithm of the machine learning model may be at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

The term “processing unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing operations of a computer or system, and/or, generally, to a device which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may be configured for machine learning. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The processing unit may be configured for pre-processing the input data. The pre-processing may comprise at least one filtering process for input data fulfilling at least one quality criterion. For example, the input data may be filtered to remove missing variables. For example, the pre-processing may comprise excluding data from subjects with less than a pre-defined minimum number of observations.

The term “training data set” as used herein is a broad term and is to be given its ordinary and customary meaning to a person of ordinary skill in the art and is not to be limited to a special or customized meaning. The term specifically may refer, without limitation, to a subset of the input data used for training the machine learning model.

The term “test data set” as used herein is a broad term and is to be given its ordinary and customary meaning to a person of ordinary skill in the art and is not to be limited to a special or customized meaning. The term specifically may refer, without limitation, to another subset of the input data used for testing the trained machine learning model. The training data set may comprise a plurality of training data sets. In particular, the training data set comprises a training data set per subject of the input data. The test data set may comprise a plurality of test data sets. In particular, the test data set comprises a test data set per subject of the input data. The processing unit may be configured for generating and/or creating per subject of the input data a training data set and a test data set, wherein the test data set per subject may comprise data only of that subject, whereas the training data set for that subject comprises all other input data.

The processing unit may be configured for performing at least one data aggregation and/or data transformation on both of the training data set and the test data set for each subject. The transformation and feature ranking steps may be performed without splitting into training data set and test data set. This may allow to enable interference of e.g. important feature from the data.

The processing unit may be configured for one or more of at least one stabilizing transformation; at least one aggregation; and at least one normalization for the training data set and for the test data set.

For example, the processing unit may be configured for subject-wise data aggregation of both of the training data set and the test data set, wherein a mean value of the features is determined for each subject.

For example, the processing unit may be configured for variance stabilization, wherein for each feature at least one variance stabilizing function is applied. The variance stabilizing function may be at least one function selected from the group consisting of: a logistic, which may be used if all values are greater 300 and no values are between 0 and 1; a logit, which may be used if all values are between 0 and 1, inclusive; a sigmoid; a log 10, which may be used if considered when all values >=0. The processing unit may be configured for transforming values of each feature using each of the variance transformation functions. The processing unit may be configured for evaluating each of the resulting distributions, including the original one, using a certain criterion. In case of a classification model as analysis model, i.e. when the target variable is discrete, said criterion may be to what extent the obtained values are able to separate the different classes. Specifically, the maximum of all class-wise mean silhouette values may be used for this end. In case of a regression model as analysis model, the criterion may be a mean absolute error obtained after regression of values, which were obtained by applying the variance stabilizing function, against the target variable. Using this selection criterion, processing unit may be configured for determining the best possible transformation, if any are better than the original values, on the training data set. The best possible transformation can be subsequently applied to the test data set.

For example, the processing unit may be configured for z-score transformation, wherein for each transformed feature the mean and standard deviations are determined on the training data set, wherein these values are used for z-score transformation on both the training data set and the test data set.

For example, the processing unit may be configured for performing three data transformation steps on both the training data set and the test data set, wherein the transformation steps comprise: 1. subject-wise data aggregation; 2. variance stabilization; 3. z-score transformation.

The processing unit may be configured for determining and/or providing at least one output of the ranking and transformation steps. For example, the output of the ranking and transformation steps may comprise at least one diagnostics plots. The diagnostics plot may comprise at least one principal component analysis (PCA) plot and/or at least one pair plot comparing key statistics related to the ranking procedure.

The processing unit is configured for determining the analysis model by training the machine learning model with the training data set. The term “training the machine learning model” as used herein is a broad term and is to be given its ordinary and customary meaning to a person of ordinary skill in the art and is not to be limited to a special or customized meaning. The term specifically may refer, without limitation, to a process of determining parameters of the algorithm of machine learning model on the training data set. The training may comprise at least one optimization or tuning process, wherein a best parameter combination is determined. The training may be performed iteratively on the training data sets of different subjects. The processing unit may be configured for considering different numbers of features for determining the analysis model by training the machine learning model with the training data set. The algorithm of the machine learning model may be applied to the training data set using a different number of features, e.g. depending on their ranking. The training may comprise n-fold cross validation to get a robust estimate of the model parameters. The training of the machine learning model may comprise at least one controlled learning process, wherein at least one hyper-parameter is chosen to control the training process. If necessary the training is step is repeated to test different combinations of hyperparameters.

In particular, subsequent to the training of the machine learning model, the processing unit is configured for predicting the target variable on the test data set using the determined analysis model. The term “determined analysis model” as used herein is a broad term and is to be given its ordinary and customary meaning to a person of ordinary skill in the art and is not to be limited to a special or customized meaning. The term specifically may refer, without limitation, to the trained machine learning model. The processing unit may be configured for predicting the target variable for each subject based on the test data set of that subject using the determined analysis model. The processing unit may be configured for predicting the target variable for each subject on the respective training and test data sets using the analysis model. The processing unit may be configured for recording and/or storing both the predicted target variable per subject and the true value of the target variable per subject, for example, in at least one output file. The term “true value of the target variable” as used herein is a broad term and is to be given its ordinary and customary meaning to a person of ordinary skill in the art and is not to be limited to a special or customized meaning. The term specifically may refer, without limitation, to the real or actual value of the target variable of that subject, which may be determined from the target data of that subject.

The processing unit is configured for determining performance of the determined analysis model based on the predicted target variable and the true value of the target variable of the test data set. The term “performance” as used herein is a broad term and is to be given its ordinary and customary meaning to a person of ordinary skill in the art and is not to be limited to a special or customized meaning. The term specifically may refer, without limitation, to suitability of the determined analysis model for predicting the target variable. The performance may be characterized by deviations between predicted target variable and true value of the target variable. The machine learning system may comprise at least one output interface. The output interface may be designed identical to the communication interface and/or may be formed integral with the communication interface. The output interface may be configured for providing at least one output. The output may comprise at least one information about the performance of the determined analysis model. The information about the performance of the determined analysis model may comprises one or more of at least one scoring chart, at least one predictions plot, at least one correlations plot, and at least one residuals plot.

The model unit may comprise a plurality of machine learning models, wherein the machine learning models are distinguished by their algorithm. For example, for building a regression model the model unit may comprise the following algorithms k nearest neighbors (kNN), linear regression, partial last-squares (PLS), random forest (RF), and extremely randomized Trees (XT). For example, for building a classification model the model unit may comprise the following algorithms k nearest neighbors (kNN), support vector machines (SVM), linear discriminant analysis (LDA), quadratic discriminant analysis (QDA), naïve Bayes (NB), random forest (RF), and extremely randomized Trees (XT). The processing unit may be configured for determining a analysis model for each of the machine learning models by training the respective machine learning model with the training data set and for predicting the target variables on the test data set using the determined analysis models.

The processing unit may be configured for determining performance of each of the determined analysis models based on the predicted target variables and the true value of the target variable of the test data set. In case of building a regression model, the output provided by the processing unit may comprise one or more of at least one scoring chart, at least one predictions plot, at least one correlations plot, and at least one residuals plot. The scoring chart may be a box plot depicting for each subject a mean absolute error from both the test and training data set and for each type of regressor, i.e. the algorithm which was used, and number of features selected. The predictions plot may show for each combination of regressor type and number of features, how well the predicted values of the target variable correlate with the true value, for both the test and the training data. The correlations plot may show the Spearman correlation coefficient between the predicted and true target variables, for each regressor type, as a function of the number of features included in the model. The residuals plot may show the correlation between the predicted target variable and the residual for each combination of regressor type and number of features, and for both the test and training data. The processing unit may be configured for determining the analysis model having the best performance, in particular based on the output.

In case of building a classification model, the output provided by the processing unit may comprise the scoring chart, showing in a box plot for each subject the mean F1 performance score, also denoted as F-score or F-measure, from both the test and training data and for each type of regressor and number of features selected. The processing unit may be configured for determining the analysis model having the best performance, in particular based on the output.

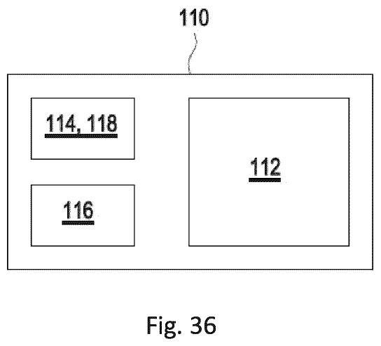

FIG. 36 shows highly schematically an embodiment of a machine learning system 110 for generating an analytical model for tracking or predicting the progression of a neurological impairment.

The analytical model may be a mathematical model configured for predicting at least one target variable for at least one state variable. The analysis model may be a regression model or a classification model. The regression model may be an analysis model comprising at least one supervised learning algorithm having as output a numerical value within a range. The classification model may be an analysis model comprising at least one supervised learning algorithm having as output a classifier such as “ill” or “healthy”.

The target variable value which is to be predicted may dependent on the disease whose presence or status is to be predicted. The target variable may be either numerical or categorical. For example, the target variable may be categorical and may be “positive” in case of presence of disease or “negative” in case of absence of the disease. The disease status may be a health condition and/or a medical condition and/or a disease stage. For example, the disease status may be healthy or ill and/or presence or absence of disease. For example, the disease status may be a value relating to a scale indicative of disease stage. The target variable may be numerical such as at least one value and/or scale value. The target variable may directly relate to the disease status and/or may indirectly relate to the disease status. For example, the target variable may need further analysis and/or processing for deriving the disease status. For example, the target variable may be a value which need to be compared to a table and/or lookup table for determine the disease status.

The machine learning system 110 comprises at least one processing unit 112 such as a processor, microprocessor, or computer system configured for machine learning, in particular for executing a logic in a given algorithm. The machine learning system 110 may be configured for performing and/or executing at least one machine learning algorithm, wherein the machine learning algorithm is configured for building the at least one analysis model based on the training data. The processing unit 112 may comprise at least one processor. In particular, the processing unit 112 may be configured for processing basic instructions that drive the computer or system. As an example, the processing unit 112 may comprise at least one arithmetic logic unit (ALU), at least one floating-point unit (FPU), such as a math coprocessor or a numeric coprocessor, a plurality of registers and a memory, such as a cache memory. In particular, the processing unit 112 may be a multi-core processor. The processing unit 112 may be configured for machine learning. The processing unit 112 may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The machine learning system comprises at least one communication interface 114 configured for receiving input data. The communication interface 114 may be configured for transferring information from a computational device, e.g. a computer, such as to send or output information, e.g. onto another device. Additionally or alternatively, the communication interface 114 may be configured for transferring information onto a computational device, e.g. onto a computer, such as to receive information. The communication interface 114 may specifically provide means for transferring or exchanging information. In particular, the communication interface 114 may provide a data transfer connection, e.g. Bluetooth, NFC, inductive coupling or the like. As an example, the communication interface 114 may be or may comprise at least one port comprising one or more of a network or internet port, a USB-port and a disk drive. The communication interface 114 may be at least one web interface.

The input data comprises a set of historical digital biomarker feature data, wherein the set of historical digital biomarker feature data comprises a plurality of measured values indicative of the disease status to be predicted. The set of historical digital biomarker feature data comprises a plurality of measured values per subject indicative of the disease status to be predicted. For example, for model building for predicting at least one target indicative of multiple sclerosis the digital biomarker feature data may be data from Floodlight POC study. For example, for model building for predicting at least one target indicative of spinal muscular atrophy the digital biomarker feature data may be data from OLEOS study. For example, for model building for predicting at least one target indicative of Huntington's disease the digital biomarker feature data may be data from HD OLE study, ISIS 44319-CS2. The input data may be determined in at least one active test and/or in at least one passive monitoring. For example, the input data may be determined in an active test using at least one mobile device such as at least one cognition test and/or at least one hand motor function test and/or or at least one mobility test.

The input data further may comprise target data. The target data comprises clinical values to predict, in particular one clinical value per subject. The target data may be either numerical or categorical. The clinical value may directly or indirectly refer to the status of the disease.

The processing unit 112 may be configured for extracting features from the input data. The extracting of features may comprise one or more of data aggregation, data reduction, data transformation and the like. The processing unit 112 may be configured for ranking the features. For example, the features may be ranked with respect to their relevance, i.e. with respect to correlation with the target variable, and/or the features may be ranked with respect to redundancy, i.e. with respect to correlation between features. The processing unit 110 may be configured for ranking the features by using a maximum-relevance-minimum-redundancy technique. This method ranks all features using a trade-off between relevance and redundancy. Specifically, the feature selection and ranking may be performed as described in Ding C., Peng H. “Minimum redundancy feature selection from microarray gene expression data”, J Bioinform Comput Biol. 2005 April; 3 (2): 185-205, PubMed PMID: 15852500. The feature selection and ranking may be performed by using a modified method compared to the method described in Ding et al., The maximum correlation coefficient may be used rather than the mean correlation coefficient and an addition transformation may be applied to it. In case of a regression model as analysis model the transformation the value of the mean correlation coefficient may be raised to the 5th power. In case of a classification model as analysis model the value of the mean correlation coefficient may be multiplied by 10.

The machine learning system 110 comprises at least one model unit 116 comprising at least one machine learning model comprising at least one algorithm. The model unit 116 may comprise a plurality of machine learning models, e.g. different machine learning models for building the regression model and machine learning models for building the classification model. For example, the analysis model may be a regression model and the algorithm of the machine learning model may be at least one algorithm selected from the group consisting of: k nearest neighbors (kNN); linear regression; partial last-squares (PLS); random forest (RF); and extremely randomized Trees (XT). For example, the analysis model may be a classification model and the algorithm of the machine learning model may be at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

The processing unit 112 may be configured for pre-processing the input data. The pre-processing 112 may comprise at least one filtering process for input data fulfilling at least one quality criterion. For example, the input data may be filtered to remove missing variables. For example, the pre-processing may comprise excluding data from subjects with less than a pre-defined minimum number of observations.

The processing unit 112 is configured for determining at least one training data set and at least one test data set from the input data set. The training data set may comprise a plurality of training data sets. In particular, the training data set comprises a training data set per subject of the input data. The test data set may comprise a plurality of test data sets. In particular, the test data set comprises a test data set per subject of the input data. The processing unit 112 may be configured for generating and/or creating per subject of the input data a training data set and a test data set, wherein the test data set per subject may comprise data only of that subject, whereas the training data set for that subject comprises all other input data.

The processing unit 112 may be configured for performing at least one data aggregation and/or data transformation on both of the training data set and the test data set for each subject. The transformation and feature ranking steps may be performed without splitting into training data set and test data set. This may allow to enable interference of e.g. important feature from the data. The processing unit 112 may be configured for one or more of at least one stabilizing transformation; at least one aggregation; and at least one normalization for the training data set and for the test data set. For example, the processing unit 112 may be configured for subject-wise data aggregation of both of the training data set and the test data set, wherein a mean value of the features is determined for each subject. For example, the processing unit 112 may be configured for variance stabilization, wherein for each feature at least one variance stabilizing function is applied. The variance stabilizing function may be at least one function selected from the group consisting of: a logistic, which may be used if all values are greater 300 and no values are between 0 and 1; a logit, which may be used if all values are between 0 and 1, inclusive; a sigmoid; a log 10, which may be used if considered when all values >=0. The processing unit 112 may be configured for transforming values of each feature using each of the variance transformation functions. The processing unit 112 may be configured for evaluating each of the resulting distributions, including the original one, using a certain criterion. In case of a classification model as analysis model, i.e. when the target variable is discrete, said criterion may be to what extent the obtained values are able to separate the different classes. Specifically, the maximum of all class-wise mean silhouette values may be used for this end. In case of a regression model as analysis model, the criterion may be a mean absolute error obtained after regression of values, which were obtained by applying the variance stabilizing function, against the target variable. Using this selection criterion, processing unit 112 may be configured for determining the best possible transformation, if any are better than the original values, on the training data set. The best possible transformation can be subsequently applied to the test data set. For example, the processing unit 112 may be configured for z-score transformation, wherein for each transformed feature the mean and standard deviations are determined on the training data set, wherein these values are used for z-score transformation on both the training data set and the test data set. For example, the processing unit 112 may be configured for performing three data transformation steps on both the training data set and the test data set, wherein the transformation steps comprise: 1. subject-wise data aggregation; 2. variance stabilization; 3. z-score transformation. The processing unit 112 may be configured for determining and/or providing at least one output of the ranking and transformation steps. For example, the output of the ranking and transformation steps may comprise at least one diagnostics plots. The diagnostics plot may comprise at least one principal component analysis (PCA) plot and/or at least one pair plot comparing key statistics related to the ranking procedure.

The processing unit 112 is configured for determining the analysis model by training the machine learning model with the training data set. The training may comprise at least one optimization or tuning process, wherein a best parameter combination is determined. The training may be performed iteratively on the training data sets of different subjects. The processing unit 112 may be configured for considering different numbers of features for determining the analysis model by training the machine learning model with the training data set. The algorithm of the machine learning model may be applied to the training data set using a different number of features, e.g. depending on their ranking. The training may comprise n-fold cross validation to get a robust estimate of the model parameters. The training of the machine learning model may comprise at least one controlled learning process, wherein at least one hyper-parameter is chosen to control the training process. If necessary the training is step is repeated to test different combinations of hyper-parameters.

In particular subsequent to the training of the machine learning model, the processing unit 112 is configured for predicting the target variable on the test data set using the determined analysis model. The processing unit 112 may be configured for predicting the target variable for each subject based on the test data set of that subject using the determined analysis model. The processing unit 112 may be configured for predicting the target variable for each subject on the respective training and test data sets using the analysis model. The processing unit 112 may be configured for recording and/or storing both the predicted target variable per subject and the true value of the target variable per subject, for example, in at least one output file.

The processing unit 112 is configured for determining performance of the determined analysis model based on the predicted target variable and the true value of the target variable of the test data set. The performance may be characterized by deviations between predicted target variable and true value of the target variable. The machine learning system 110 may comprises at least one output interface 118. The output interface 118 may be designed identical to the communication interface 114 and/or may be formed integral with the communication interface 114. The output interface 118 may be configured for providing at least one output. The output may comprise at least one information about the performance of the determined analysis model. The information about the performance of the determined analysis model may comprises one or more of at least one scoring chart, at least one predictions plot, at least one correlations plot, and at least one residuals plot.

The model unit 116 may comprise a plurality of machine learning models, wherein the machine learning models are distinguished by their algorithm. For example, for building a regression model the model unit 116 may comprise the following algorithms k nearest neighbors (kNN), linear regression, partial last-squares (PLS), random forest (RF), and extremely randomized Trees (XT). For example, for building a classification model the model unit 116 may comprise the following algorithms k nearest neighbors (kNN), support vector machines (SVM), linear discriminant analysis (LDA), quadratic discriminant analysis (QDA), naïve Bayes (NB), random forest (RF), and extremely randomized Trees (XT). The processing unit 112 may be configured for determining a analysis model for each of the machine learning models by training the respective machine learning model with the training data set and for predicting the target variables on the test data set using the determined analysis models.

### EXAMPLES

### Draw-a-Shape Test

The inventors demonstrated the principles of the invention using data from patients performing a Draw-a-shape test.

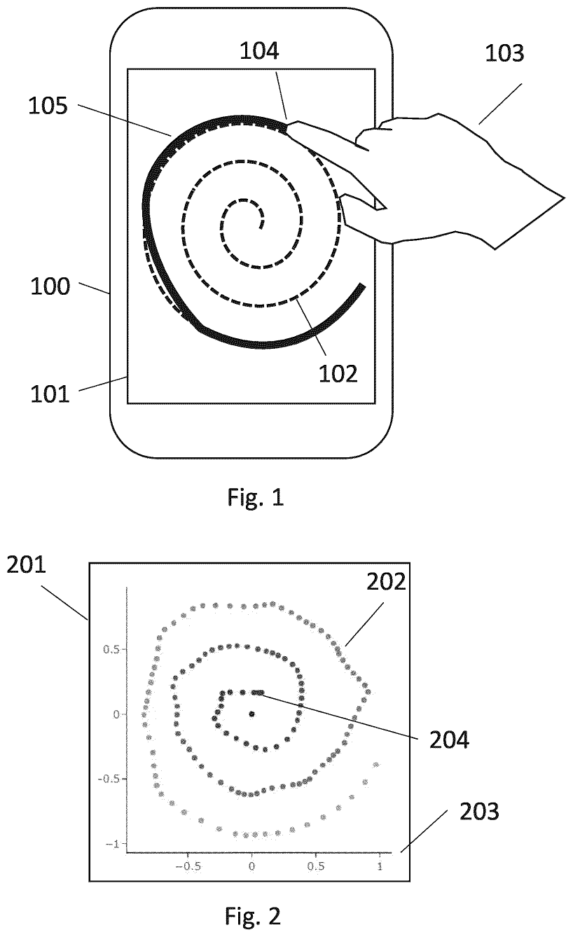

FIG. 1 displays a schematic view of a patient performing the Draw-a-Shape (DAS) test. The DAS test is used as a test of hand motor function, which can be indicative of state of health of the patient, particularly with respect to diseases such as MS.

In the test, a device 100, such as a smartphone, having a touch screen 101, such as a smartphone, is used to display an image of a desired shape 102. The patient is then requested to trace the desired shape, using their hand 103, preferably their finger.

Ideally, the patient would move their hand 103 such that they move their point of contact 104 with the touch screen along the desired path 102 of the shape exactly. As the patient moves their point of contact 104 along the desired path 102, the touch screen device 101 records the point of contact 104 throughout the patient's attempt to trace the shape to produce a recorded path 105. This recorded path 105 can be displayed to the patient during the attempt in order to allow them to see the deviation between the desired path 102 and the recorded path 105. This can allow the patient to make corrections during the attempt so that they can more accurately reproduce the desired path 102.

Typically, the patient is asked to trace a variety a shape repeatedly, either until a satisfactory number of recorded paths are obtained or until the patient fatigues. The shape can be a variety of shapes such as a spiral (as shown in FIG. 1), a circle, a square, a triangle, a figure-of-eight (8) or any other desired shape.

The DAS test is described in greater detail in Creagh et al (2020) Smartphone-based remote assessment of upper extremity function for multiple sclerosis using the Draw a Shape Test. Physiological Measurement, 41 (5), 054002.

FIG. 2 displays an example data set 201 obtained from a patient's attempt at the DAS test. The recorded path 105, is represented by a series of touch points 202 made at regular intervals at the point of contact 104 during the test. Each touch point is recorded as a coordinate value in the horizontal and vertical axis 203.

Multiple formats for these coordinate values were tested: a X and Y coordinate value, a series of relative coordinates (deltaXY) providing the change in position from one point to the next, and the error from the ideal shape that was made (vecXY). X and Y coordinates were ultimately chosen because it is the simplest and provided the best results.

This coordinate value is rescaled to be a value between −1 and +1 on both axes 203. A vector of 1s was added as the stroke which ends with 0s to indicate the end of the drawing. An initial value of [0, 0, 0, 0, 0, 0, 1] is added, which is used by the LSTM decoder.

Extra information (e.g. the original dataframe) is passed through to the network as it is useful for validation purposes and when training the head that predicts 9HPT times. A modified loss function which calculates the errors that the patient has made from the ideal shape and upweights this difference so that the network would focus more on encoding these offsets. This component of the loss was weighed 500x and it is annealed from 0 the same way as the KL loss weight described below. The loss calculation was of the format:
def fancy loss (Y_hat, Y, vecXY):

$$ideal\_shape = Y + vecXY$$

$$calculated\_vecXY = ideal\_shape - Y\_hat$$

$$Loss\_r = MSE ( Y\_hat , Y )$$

$$Loss\_vec = MSE ( calculated\_vecXY , vecXY )$$

$$return w\_a ⋆ Loss\_r + w\_b ⋆ Loss\_vec$$

### Autoencoders

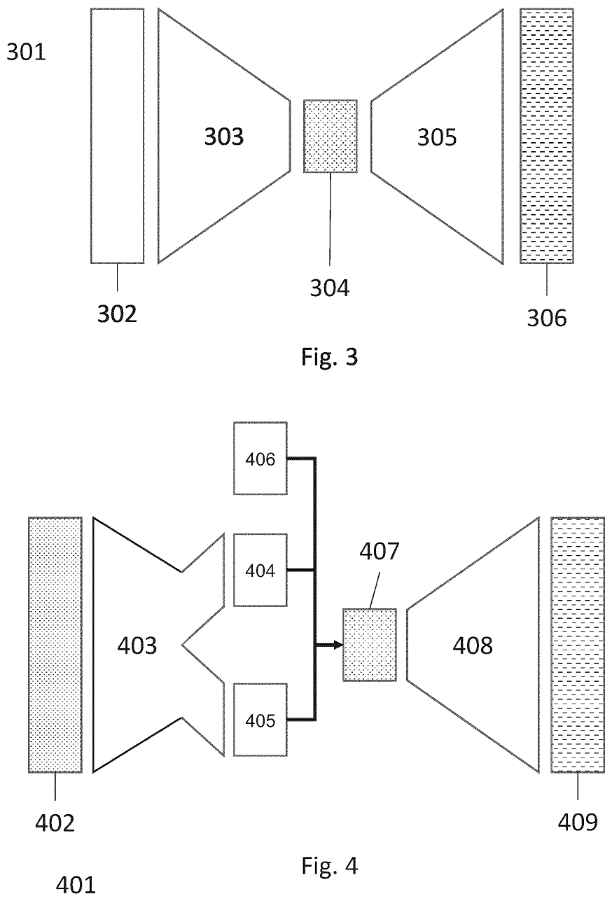

FIG. 3 displays a schematic representation of an autoencoder 301. An autoencoder 301 is a type of artificial neural network which is adapted to be trained in an unsupervised manner. At a high-level, an autoencoder may operate by transforming input data into data having a lower dimensionality, or by reducing an input data set 302 comprising a first number of variables (or other representative values or parameters) to an intermediate data set 304 comprising a second number of variables (or other representative values or parameters), the second number less than the first number. These variables may be referred to as “latent variables”. Then, the autoencoder may be configured to transform the intermediate data set 304 to an output data set 306, the output data set comprising a third number of variables (or other representative values or parameters), wherein the third number is greater than the second number. In some cases, the third number may be the same as the first number.

In this manner, an autoencoder is an analytical model, the purpose of which is to reconstruct an input data set by reducing its dimensionality (referred to herein as “encoding”), and then by increasing the dimensionality of the data set again (referred to herein as “decoding”). Thus, the autoencoder effectively identifies, within the data set, variables or patterns which best represent the main trends or features of the data, and which allow the data best to be reconstructed. The VAE framework is described in detail in Kingma, D. P., & Welling, M. (2013). Auto-Encoding Variational Bayes (http://arxiv.org/abs/1312.6114v10).

Accordingly, the autoencoder comprises an encoder 303 (or equivalently, an encoder layer) configured to receive an input data set 302 comprising a first number of variables and to transform the input data set 302 into an intermediate data set 304 comprising a second number of variables, wherein the second number is less than the first number. The autoencoder further comprises a decoder 305 (or alternatively, a decoder layer) configured to receive the intermediate data set 304 comprising the second number of variables and to transform the intermediate set 304 of variables to an output data set 306 comprising a third number of variables, wherein the third number is greater than the second number. As discussed, the third number may be equal to the first number.

The intermediate data set 304 can be referred to as the latent space or the bottleneck.

Alternatively put, the encoder layer 303 may be configured to receive an input in the form of an N-dimensional vector, and to map the input into an M-dimensional array, wherein M is less than N. And, the decoder layer 305 may be configured to receive the intermediate M-dimensional array, and to reconstruct a P-dimensional output vector. N may be equal to P.

FIG. 4 displays a schematic representation of a variational autoencoder (VAE) 401. A variational autoencoder is a type of autoencoder and generally serves the same purpose. However, autoencoders are typically deterministic in their encoding and decoding. This can lead to overfitting as the analytical model attempts to minimise the reconstruction error of the encoder and decoder to exactly match the training data. This can be conceptualised as the latent space being highly irregular. The outcome of this is that when new input data, outside of the training data set, is applied to the model, it does not produce the expected output data. Variational autoencoders sample a parametrised distribution of the training data in order to regularise the latent space. This makes at least the encoder of the autoencoder non-deterministic, with the result that the latent space is more regular and can handle new input data. The VAE is described in detail in Kingma & Welling (ibid).

Like the standard autoencoder, a variational autoencoder is also an analytical model. Whereas the purpose of the regular encoder was to reconstruct an original input, the purpose of a variational encoder is to identify a probability distribution which can be used to recreate inputs having similar characteristics to the input data set.

The variational autoencoder 401 may operate by transforming input data into data having a lower dimensionality, or by reducing an input data set 402 comprising a first number of variables (or other representative values or parameters) to an intermediate data set 407 comprising a second number of variables, as with a regular autoencoder.

Then, unlike the standard autoencoder 301, the variational encoder 401 may be configured to determine the mean 404 and standard deviation 405 of each of the latent variables. This operates on the assumption that the spread of the values of each latent variable obeys a predetermined probability distribution (herein, the “latent distribution”, preferably a normal distribution. Other statistical measures may be determined instead of the mean and standard deviation, e.g. the variance.

After the mean 404 and standard deviation 405 have been determined, the variational autoencoder may be configured to generate a value for each latent variable by sampling each latent distribution. In this way, an intermediate latent vector 407 may be generated, the latent vector comprising a second number of variables. The variational decoder 408 may then be configured to transform the latent vector 407 comprising the second number of variables into an output data set 409 comprising the third number of variables (or other representative values or parameters), wherein the third number is greater than the second number.

### Architectures

Long Short-Term Memory (LSTM) cells

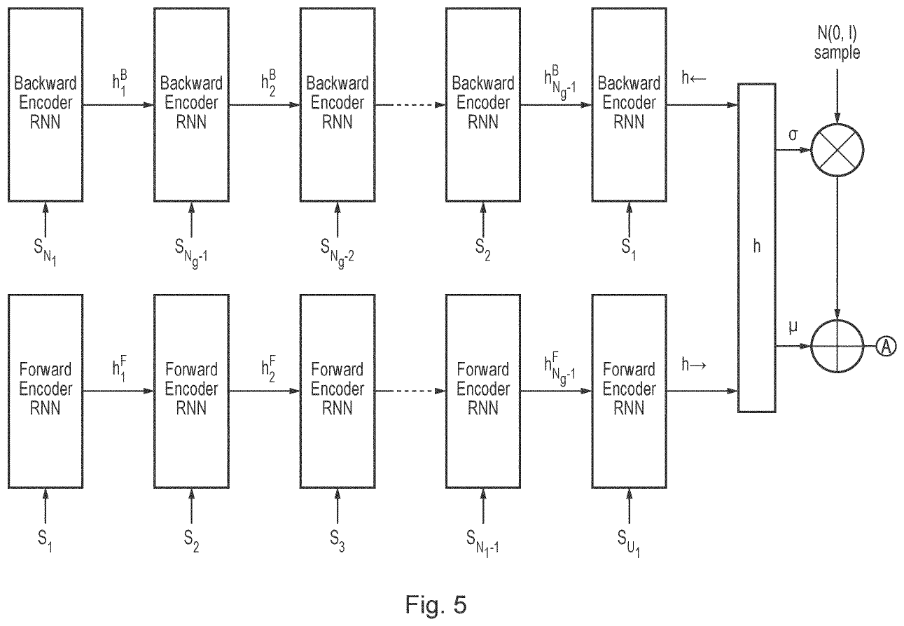

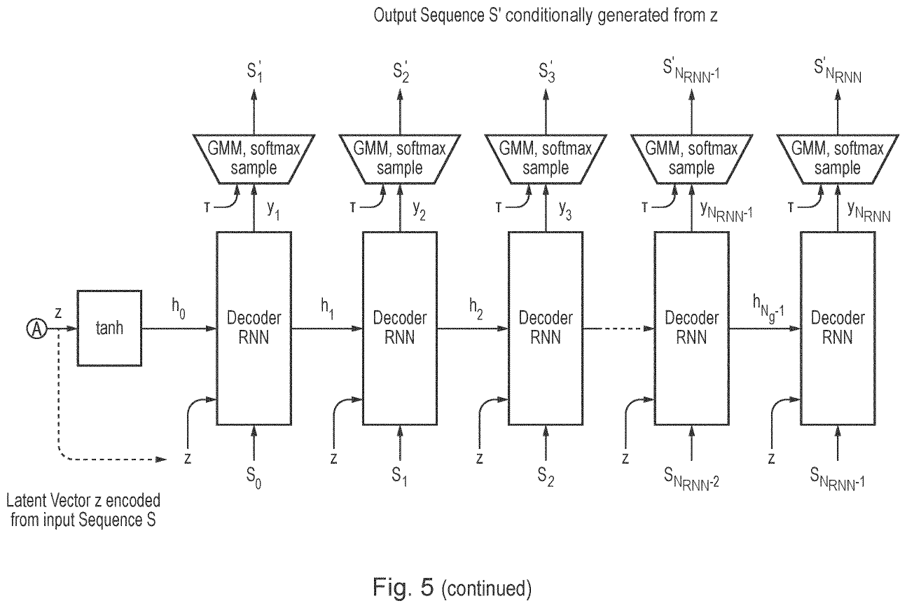

FIG. 5 displays a recurrent architecture that uses Long Short-Term Memory (LSTM) cells which can be used as a bidirectional encoder and a normal recurrent encoder. This architecture is that of sketch-RNN as used in Ha and Eck (2017) “A Neural Representation of Sketch Drawings” (https://arxiv.org/abs/1704.03477) and this figure is derived from FIG. 2 thereof. The encoder network is implemented as an optionally bidirectional and optionally multi-layer LSTM network. The decoder network is implemented as a single layer unidirectional LSTM network. Three different LSTM block variants are implemented: LSTM, Layer Normalized LSTM, (which adds layer normalization at each step) see Ba et al. (2016) “Layer Normalisation” (https://arxiv.org/abs/1607.06450), and HyperLSTM (which uses an auxiliary network to determine the weight of the real network, see Ha et al. (2016)) “HyperNetworks” (http://arxiv.org/abs/1609.09106).

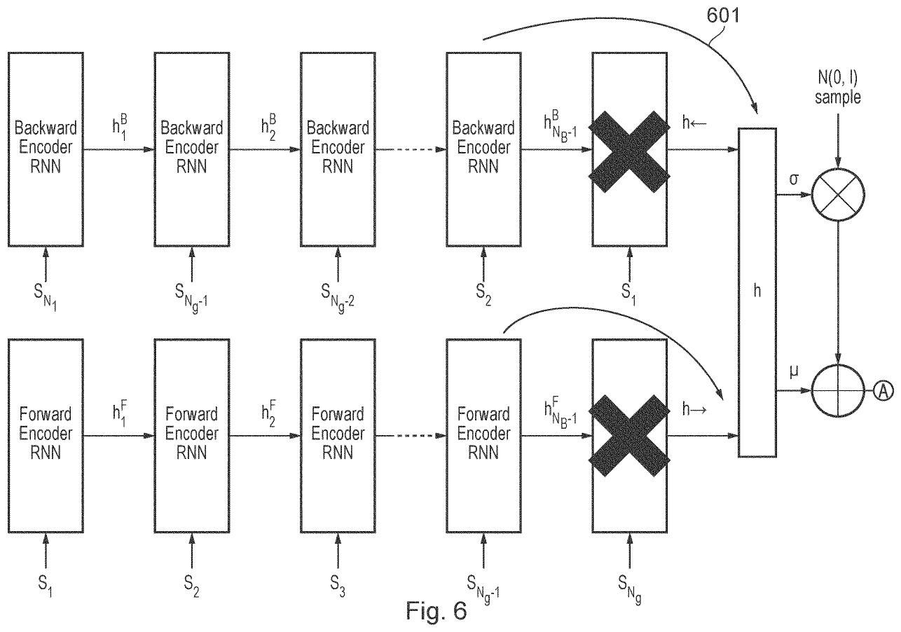

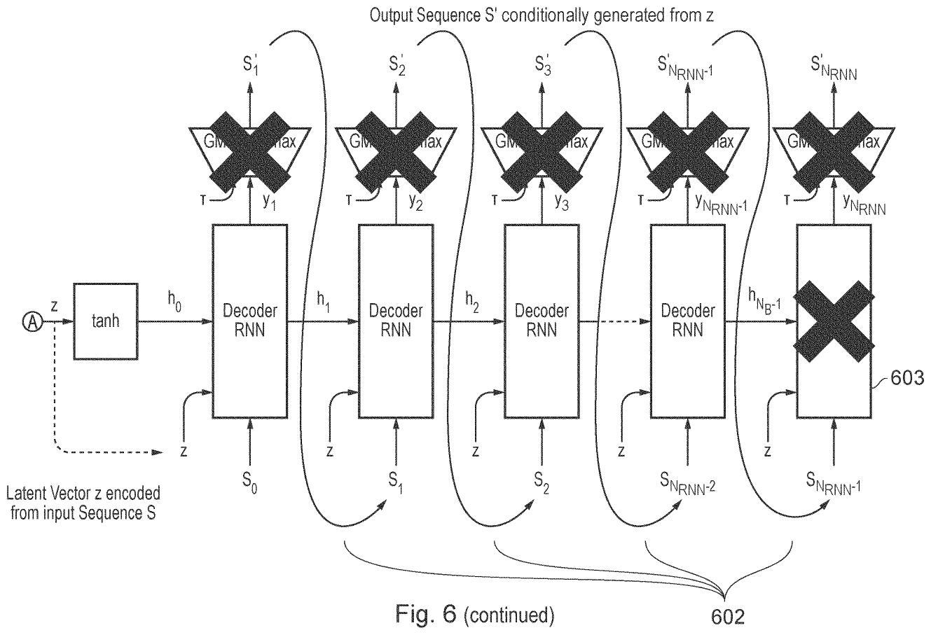

FIG. 6 displays a recurrent architecture that has been modified from the architecture displayed in FIG. 5. Modifications were made to the architecture from Ha and Eck (ibid) to make it more compatible with the training data of the present application.

Sequence masking was added to the sketch RNN architecture. This means that for each sample the encoder skips the steps after the end of FIG. 5 (by means of indexing the correct hidden state) and in the decoder steps after the ending of the drawing are ignored.

The Gaussian mixture model network (603) that is appended on top of the decoder network, which is supposed to sample from distributions rather than directly output the values, was completed disregarded for simplicity.

Scheduled learning was added in the decoder portion (602), in which the decoder network is fed its own outputs from the previous time steps rather than the input data, as described in Bengio et al (2015) “Scheduled Sampling for Sequence Prediction with Recurrent Neural Networks” (https://arxiv.org/abs/1506.03099).

Hyperparameter optimisation was used to determine the best architecture to use. Masking the values for the network significantly improves the exploding gradient problems and allows the use of more complex architectures such as multilayer stacked LSTMS. Moreover, scheduled learning was seen to significantly improve reconstructions at test time. The cell type did not make significant differences in the reconstruction quality, whilst having a bidirectional stacked encoder helped as well as having larger hidden dimensions. Dropout and recurrent dropout did not have significant impact on reconstruction.

The final model uses Layer Normalised LSTM cells for both encoder and decoder. The encoder is a 2 layer stacked and bidirectional architecture. The hidden size was 512 for the encoder and 1024 for the decoder cells. The encoder has 8.5M parameters and the decoder 4.4M, for a total of 12.8M parameters.

### Convolutional Neural Network

An alternative neural network architecture that was used for encoding decoding was a 1 dimensional convolutional neural network (CNN) based on the architecture described in Krizhevsky et al (2012) “ImageNet Classification with Deep Convolutional Neural Networks” (Advances in Neural Information Processing Systems 25) 1-Dimensional convolutions that apply a kernel over the data sequence at time steps were used. The encoder and decoder network were designed with 5 convolutional blocks that progressively bring the dimensionality of the data from 256 to 8 and back to 256 with strided and transposed convolutions. These blocks contain 1D convolutions in which the dimensionality is the time steps whilst the X, Y and stroke coordinates are features equivalent to RGB in images. The number of filters used in each block is a hyperparameter.

4 different block types were implemented:
VGG single style: a single convolutional layer per block
VGG: two convolutional layers per block
Resnet: two convolutional layers with a skip connection that uses a convolution with a kernel size of 1 to project the stride and the number of filters
Bottleneck resnet: this block first reduces the number of filters using a convolution with a kernel size of 1 and then uses two normal convolutional layers before reprojecting it up to the desired number of filters. This allows the network to have more layers with fewer parameters.

All blocks implement 4 different types of normalization:
Batch normalization
Layer normalization
Instance normalization
Weight normalization

VGG and VGG single blocks with batch normalization were found to be the best combinations. This is unexpected for variational autoencoders as in the prior art, different kinds of normalization are typically used. The momentum was specifically set to 0.99, as this has been shown to be more stable for variational autoencoders and other architectures. The CNN model contains significantly fewer parameters than the LSTM model, with 865K parameters in the encoder and 1.1M in the decoder for a total of 2M parameters.

### Approach

Data was subsampled by 3 for the LSTM network and by 2 for the CNN. This is to reduce the dimensionality of the data such that most samples within 200 points for LSTM and 256 for the CNN. Each data set of points was padded to a fixed length of 200 points for LSTM and 256 points for CNN. A length vector was also calculated and passed through for masking purposes.

The KL loss component acts to regularise the latent space, by forcing the network to balance the amount of information that passes through the latent space vs the quality of the reconstruction. Multiple papers have explored using different KL loss weights and annealings, starting with the beta-VAE paper, to enforce a higher or lower strictness on the prior.

The KL weight is a hyperparameter that is set in VAE config, and we have experimented with different weights. The basic KL loss weight that we use is always annealed from 0 to 1*KL weight with an exponential function. With an R parameter set to 0.9995 the KL loss anneals to 0.8 in about 10 epochs. This annealing strategy is presented in Bowman et al., 2015 “Generating Sentences from a Continuous Space” (http://arxiv.org/abs/1511.06349). and is used to mitigate the posterior collapse problem. This is when the decoder completely ignores the latent dimensions and predicts the mean of the training set, because it gets stuck in a local minima in which it cannot increase the KL loss without significantly reducing the reconstruction loss. This issue was not observed in our dataset.

We have implemented a cyclical KL weight anneal (cycle between 0 and 1 four times before stabilizing at 1), see Fu et al., 2019, “A Simple Approach to Mitigating KL Vanishing” (http://arxiv.org/abs/1903.10145) and a reverse anneal (start from a weight of 10 and anneals down to 1) as well, see Burgess et al., 2018, “Understanding disentangling in B-VAE” (http://arxiv.org/abs/1804.03599).

In general, the higher the KL weight, the worse and more smoothed the reconstructions are. This is because significantly less information can be passed through the bottleneck. The cyclical KL weigh anneal and reverse anneal do not seem to have significant impact on the results.

### Latent Space and Reconstruction Quality

In general, the aim with the VAE was not to create the best reconstructions or generate new data as a generative model but to create the best latent representation of the data as possible. The reconstruction quality was mainly used as a proxy to understand which information was captured in the latent space. It was assumed that the decoder would be able to fully utilise the latent space.

The reconstruction quality was evaluated both visually and with metrics. The reconstruction quality changes significantly when varying the KL loss weight term. To compare the models, the same KL loss weight was used for both but hyperparameter optimisation was done separately. Another parameter to consider is the size of the latent space. Even though the network uses only 3 or 4 dimensions, increasing the dimensionality of the bottleneck effectively reduces the KL loss weight because the KL loss is calculated as a mean over all the dimensions. The distribution of Z mean values that are produced by the encoder over a test dataset were considered in order to verify which latent dimensions are used. Because of the Gaussian normal prior, if that specific latent dimension is not used then its outputs will always be zero.

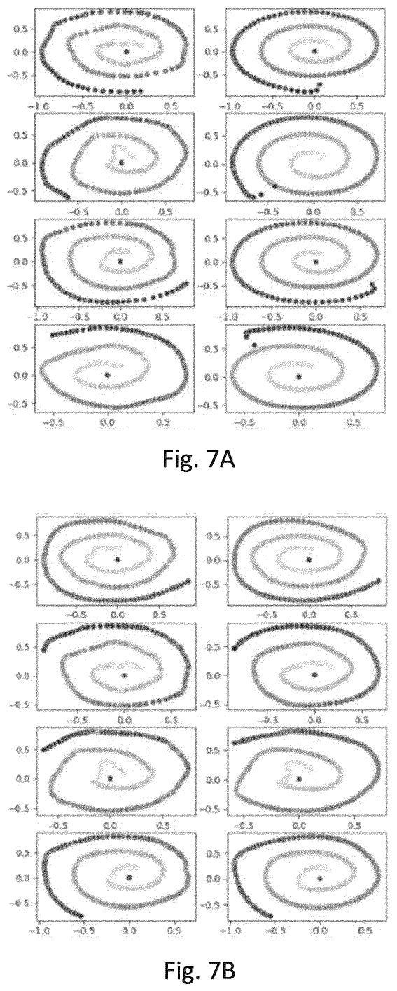

FIG. 7A displays a series of graphs of recorded path data and reconstructions of those paths by the LSTM decoder. The four graphs on the left-hand side of the figure show the original data of the recorded paths. The four graphs on the right-hand side show the output data for each of the corresponding recorded paths on the left-hand side after the recorded data has been encoded into the latent space and then decoded.

The LSTM model shows a limited capability to encode the errors that the patients make in the DAS test. The reconstructions produced by the LSTM decoder produce a smoothed reconstruction of the desired shape. However, the drawing speed and position at the end of the drawing appear to be encoded.

FIG. 7B similarly displays a series of graphs of recorded path data and reconstructions of those paths by the CNN decoder. The four graphs on the left-hand side of the figure show the original data of the recorded paths. The four graphs on the right-hand side show the output data for each of the corresponding recorded paths on the left-hand side after the recorded data has been encoded into the latent space and then decoded.

From the figure, it can be seen that the CNN model seems to reconstruct the errors in drawings made by the patient more accurately compared to the LSTM model. Finer errors were able to be maintained in the reconstruction when the KL divergence term was removed from the loss function.

This reconstruction error is reflected in the final MSE loss that we have. On consonance data:
KL0:
   0.003 for LSTM
   4.384e-4 for CNN
KL1:
   0.012 for LSTM
   1.135e-3 for CNN

The final models were tested with a KL loss weight of 0, 0.1 and 1, and the reconstruction quality progressively worsens with both CNN and LSTM models. For the final CNN model, the following reconstruction errors on consonance data were calculated.

| KL weight | Real MSE |
|---|---|
| 0 | 4.384e−4 |
| 0.1 | 5.014e−4 |
| 1 | 1.135e−3 |

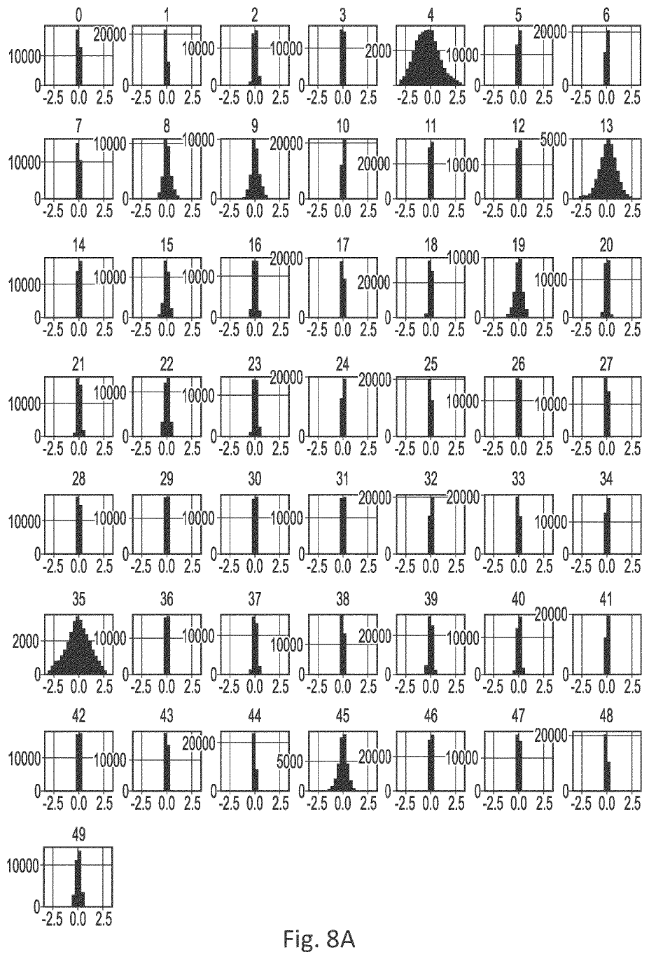

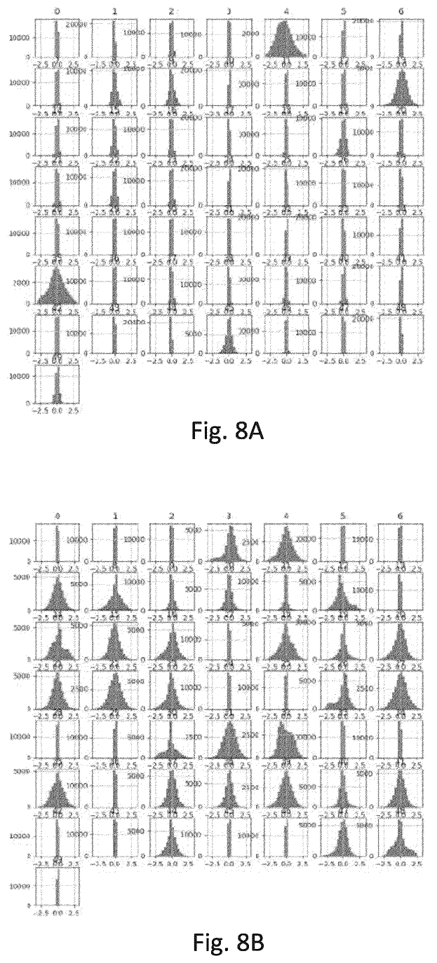

FIG. 8A is a series of graphs representing how many latent dimensions of the bottleneck layer are used in the LSTM model. FIG. 8B is a series of graphs representing how many latent dimensions of the bottleneck later is used in the CNN model.

Each small graph in FIGS. 8A and 8B represents the use of a dimension of the latent space produced by the respective architectures over a test dataset. Each graph is labelled with an index value above it. If a given dimension is not used, it should output zero (appearing as a thin spike), as the normally distributed prior used by each encoder has a zero mean.

From these graphs it can be seen that the LSTM model (FIG. 8A) uses far fewer dimensions, since most of the graphs shown simple spikes at zero midpoint, compared with that of the CNN model. The latent space of the LSTM model is therefore much simpler. This helps to explain why the CNN model has improved reconstruction quality compared to the LSTM model and why more information appears to be encoded into the latent space.

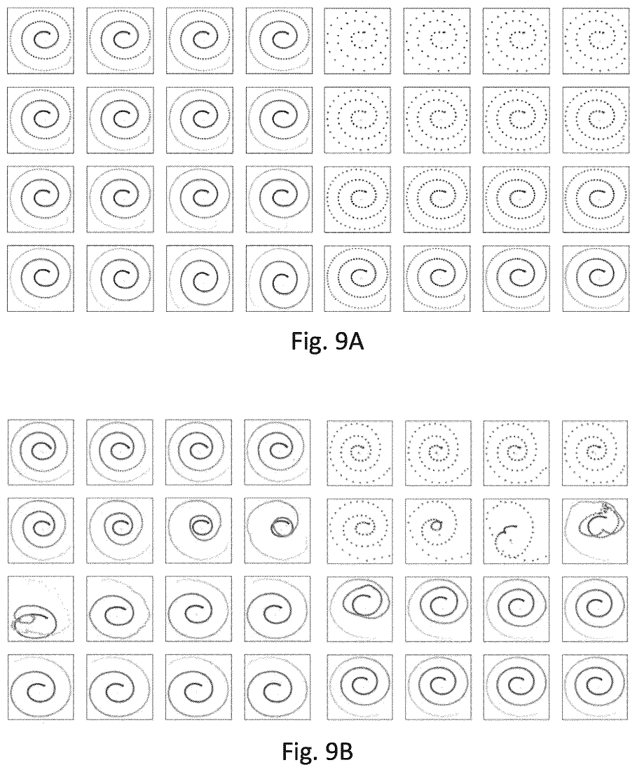

FIG. 9A is a series graphs visualising the output of generated data from the LSTM architecture. FIG. 9B is the same from the CNN architecture. The generative capacity of the LSTM and CNN decoders was tested in order to further consider what information is encoded in the latent space. To test this, the latent representation of two shapes were interpolated and the result processed by the decoder.

LSTM models only used a few latent variables. The resulting reconstructions were very smooth but encoded the drawing speed as well as the end of the spiral. So few latent dimensions were used that it was possible to identify one or two latent variables that encoded the drawing speed and individually alter it in the reconstruction.

CNN models produced significantly better reconstructions but both interpolation and latent variable independence were not preserved. When changing individual variables, the output shape would change but no single variable encoded a single feature such as the drawing speed. In some circumstances, the model output would collapse if the generated data was outside the region covered by the training dataset. Attempts using other KL annealing schedules did not seem to change these results significantly. Using a latent dimension of 4 sometimes helped whilst other it did not.

The LSTM model provides a superior generative capacity to the CNN model as the interpolated data is more accurately reconstructed by the LSTM decoder. It seems that CNNs have inherently fewer inductive biases for this kind of sequence generation, even though they can encode a lot more information, and so are not able to generalise as well as the LSTM models.

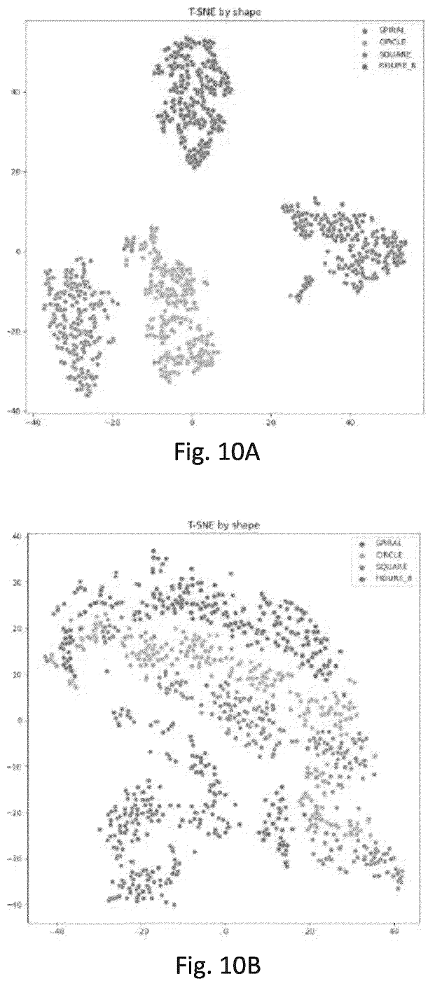

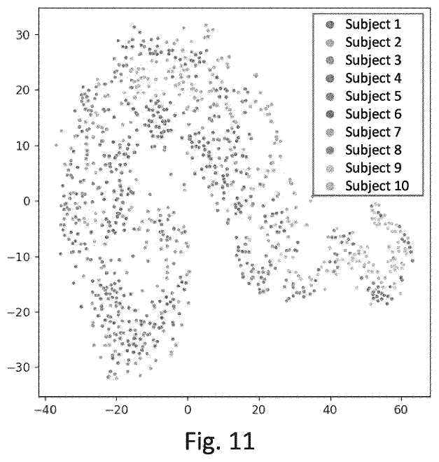

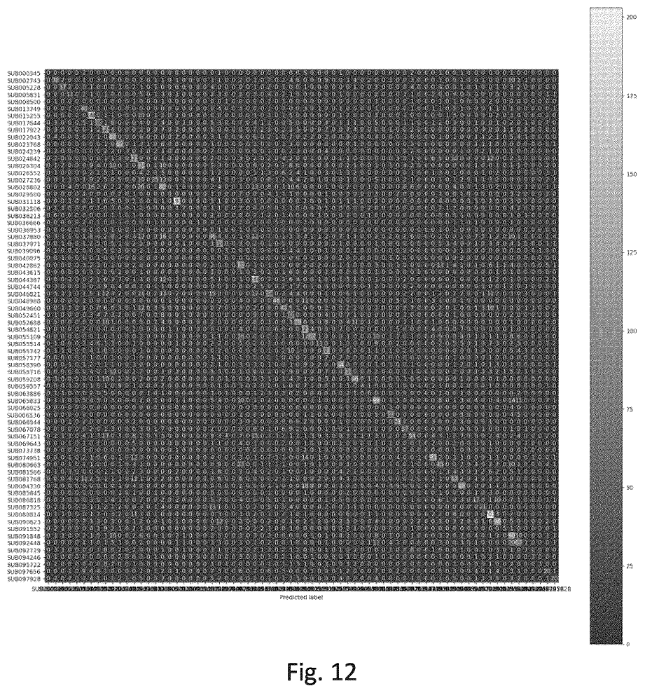

FIG. 10A is a t-distributed stochastic neighbour embedding (t-SNE) representation of the latent space of the LSTM model. FIG. 10B is the same for the CNN model. The t-SNE graph shows that the latent space for the 4 different shapes trained for (i.e. spiral, circle, square, and figure-8) are more clearly separated in the LSTM model (FIG. 10A) compared to the CNN model (FIG. 10B).

Without wishing to be bound to any specific theory of the mechanism, it is reasoned that the latent space of the CNN model is more complex. In particular, the encoded data may lie on a subspace (or manifold) of the higher dimensional space of the latent space. An interpolation between two shapes may be moving through an area where no samples exist in latent space, rather than following the manifold.

The latent space of the LSTM and CNN models were further tested and validated by testing the ability of the model to classify the subject ID using the latent space representation of each shape. Being able to classify subjects would mean that the network is encoding enough information in the latent space to identify the drawing style of the subjects. This is implemented both as a metric in the validation callback, for which only the subjects in the validation set are used, and also tested for all subjects in the Floodlight POC data (Consonance is too large for this). All the models are trained using a random train/test split with 20% used for testing. All values reported here used an SVM model on the validation set. The validation set is 18 subjects for Floodlight POC and 72 subjects for consonance. Balanced accuracy weights all subjects the same amount and was calculated using a function of the skLearn library.

Also, the way this is implemented (multiclass classification) the accuracy values depend on how many subjects are used in the test, as having more subjects significantly increases the difficulty of the task.

It is also important to note that due to the models used in the classification the latent space doesn't necessarily need to be regular, linear or with independent dimensions, as long as similar shapes reside close to each other and there are enough samples to identify the pattern for each subject. This is due to the choice of models which are very highly dimensional (KNN or SVM).

It is possible to identify the subject id with an accuracy much better than random (˜50%), which means that the subjects have inherent drawing styles that are different enough for that drawing to be uniquely classified to that subject.

Improving reconstruction quality seems to improve the metric. The KL loss weight value seems to be optimal at 0.1, probably balancing regularity of the latent shape with reconstruction quality.

TABLE
Accuracy results for subject id classification with
different models, datasets and KL loss weight.

| Model | | KL loss | | Balanced |
|---|---|---|---|---|
| type | Dataset | weight | Accuracy | Accuracy |
| CNN | Floodlight | 1 | 0.451 | 0.403 |
| | POC | | | |
| CNN | Floodlight | 0.1 | 0.508 | 0.45 |
| | POC | | | |
| CNN | Floodlight | 0 | 0.482 | 0.428 |
| | POC | | | |
| LSTM | Floodlight | 1 | 0.394 | 0.351 |
| | POC | | | |
| LSTM | Floodlight | 0.1 | 0.429 | 0.384 |
| | POC | | | |
| LSTM | Floodlight | 0 | 0.482 | 0.421 |
| | POC | | | |
| CNN | Consonance | 1 | 0.481 | 0.343 |
| CNN | Consonance | 0.1 | 0.524 | 0.38 |
| CNN | Consonance | 0 | 0.471 | 0.329 |
| LSTM | Consonance | 1 | 0.254 | 0.169 |
| LSTM | Consonance | 0.1 | 0.361 | 0.249 |
| LSTM | Consonance | 0 | 0.423 | 0.294 |

FIG. 11 shows a t-distributed stochastic neighbour embedding (t-SNE) representation of the latent space of each model for 10 random subjects taken from the Consonance data set. The points for different subjects formed separate islands within the plot. This indicates that the latent space is successfully encoding this information.

FIG. 12 shows a Confusion matrix showing the true subject labels vs predicted subject labels for the CNN model with KL1 on consonance data. Each row and column is an individual subject. This was done to show that a classifier trained on the latent space could recover the subject that drew that shape. It is an indication that different subjects have distinct drawing styles that differentiate each other

It is apparent that it is difficult to interpret the CNN models and the latent dimensions therein are not independent. Moreover, the models do not cover the whole possible latent space. This is apparent from the generated data shown in FIG. 9B. This is most likely due to the high dimensionality of the space.

Manifold discovery techniques such as t-SNE and UMAP were used to visualise the data. Moreover, non-linear methods such as mutual information and training regressors need to be used to search for what information is captured. The regressors were trained using auto-sklearn, which automatically optimises preprocessing and hyperparameter optimisation. The R² score of the predictions on a hold-out validation set is reasonable proxy for how much of that feature was encoded in the latent variables or the reverse, how much those features can reproduce the latent variable.

As KL loss and the sampling are still in the process, similar shapes should still be located close by in the latent space by some metric, even if the latent space is a contorted lower dimensionality shape that cover a small fraction of the span of the space. This can be tested by using t-SNE on the latent space.

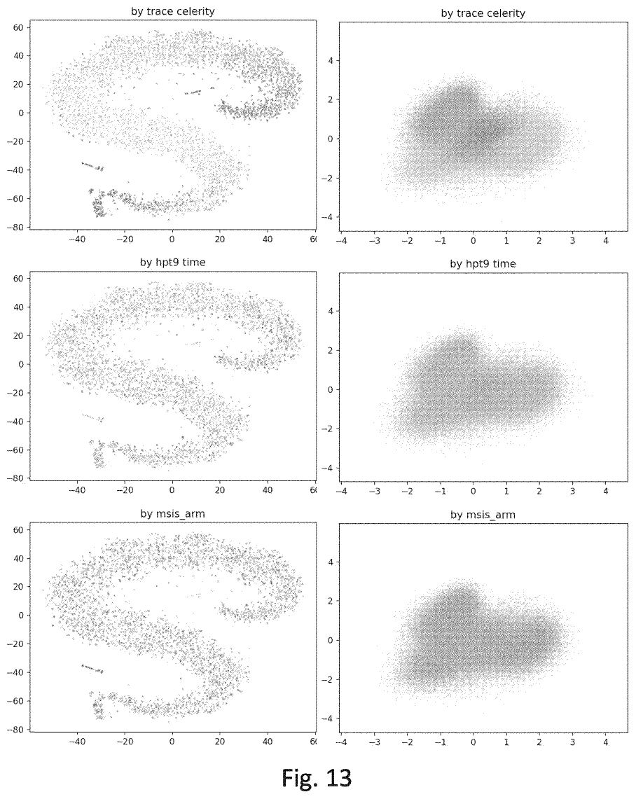

FIG. 13 shows a series of graphs. The left-hand column is a t-SNE of the data from the Consonance study and the right hand column is a PCA of the same. The top graphs on both sides are coloured by trace celerity, the middle graphs by 9HPT times, and the bottom graphs by msis_arm. msis_arm represents separate data from a questionnaire done by the patient.

These results are good and better than a simple PCA. When colouring the latent space using some of the handcrafted features it is clearly possible to see a gradient of that feature. Unfortunately, for both of these methods the resulting distance between points on the graph does not have any meaning so it is not possible to use it to calculate distances.

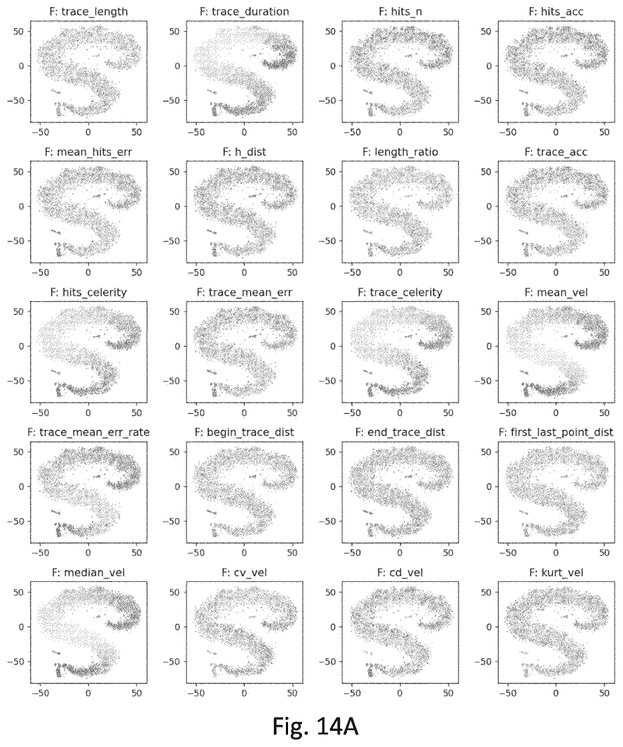

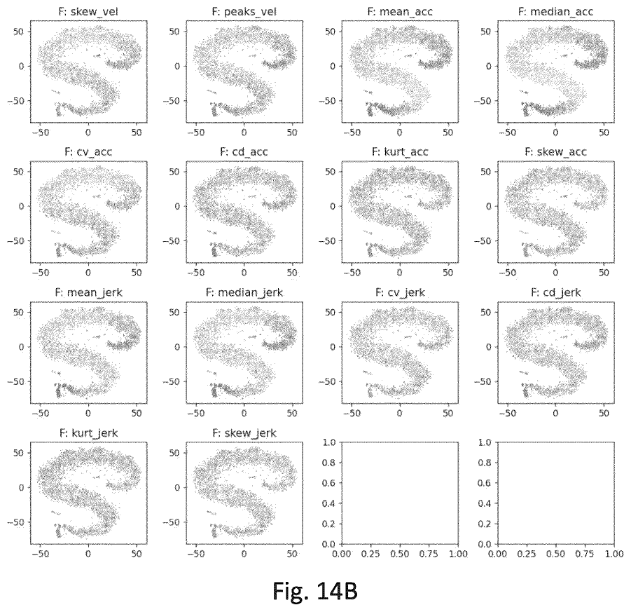

FIGS. 14A and 14B show further t-SNE representations of the Consonance data as in FIG. 13 but the points are coloured by the handcrafted feature listed above each graph. As can be seen, some features, such as trace duration, show a clear gradient. Whereas other features, such as trace length, any gradient or trend is less clear.

These plots show how the latent space is ordered according to certain handcrafted features. This raises the question of to what extent these handcrafted features are encoded in the latent variables.

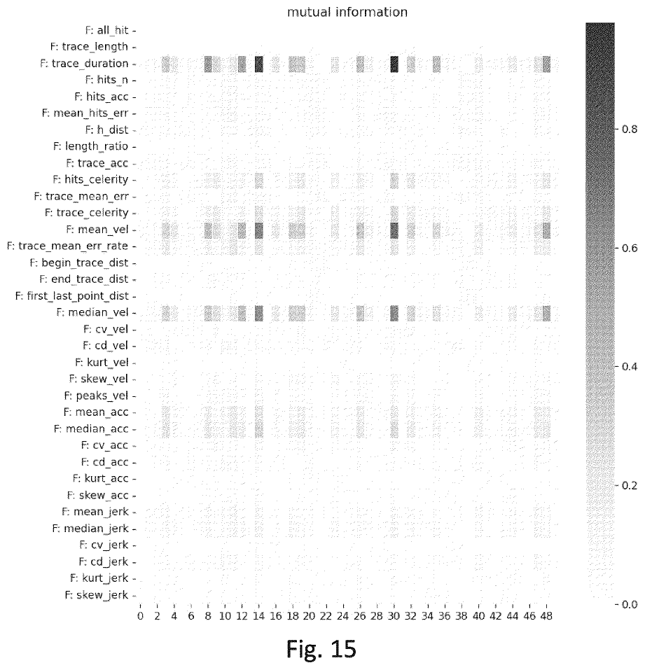

FIG. 15 shows a heatmap graph representing the level to which a set of handcrafted features are encoded in the dimensions of the CNN model. For each list handcrafted feature on the vertical axis, there is a corresponding number on the horizontal axis for each dimension. The degree of shading at given position represents the degree to which information about that handcrafted feature is encoded on that dimension, with darker representing more representation and lighter representing less representation.

As can be seen in the shading of FIG. 12, the primary features encoded in the CNN model is related to drawing speed (e.g. trace duration, mean vel, median vel). There is also some information encoded about accuracy (e.g. hits celerity, mean acc). This reflects what is seen in FIGS. 14A and 14B.

These values were calculated using a mutual information score, which is calculated using the following formula:

$$MI ( U , V ) = {\sum }_{i = 1}^{\vert U \vert} {\sum }_{j = 1}^{\vert V \vert} \frac{\vert {U}_{i} ∩ {V}_{j} \vert}{N} log \frac{N \vert {U}_{i} ∩ {V}_{j} \vert}{\vert {U}_{i} \vert \vert {V}_{j} \vert}$$

Where |Ui| is the number of the samples in cluster Ui and |Vj| is the number of the samples in cluster Vj. Mutual Information is a feature of skLearn.

The mutual information tends to be really conservative and can only produce a one to one relationship. For this reason, autosklearn was used to predict each individual handcrafted feature from all the latent values of the consonance dataset. In the table below the first column used all the latent dimensions to predict the features, whilst the second column used only the top 10 latents that were deemed important by an 9HPT predictor trained on top of the latent space (See results in section below). This is to verify if the latents that were useful for the 9HPT prediction primarily encoded the features.

The results correlate with mutual information but are different probably because some features are encoded across multiple latent variables.

R² value

| | | Top 10 |
|---|---|---|
| | All latent | dimensions for |
| Feature name | dimensions | 9-HPT prediction |
| all_hit | −0.006 | −0.013 |
| trace_length | 0.022 | 0.014 |
| trace_duration | 0.640 | 0.595 |
| hits_n | 0.336 | 0.242 |
| hits_acc | 0.336 | 0.242 |
| Mean_hits_err | 0.075 | 0.079 |
| h_dist | 0.164 | 0.110 |
| length_ratio | 0.022 | 0.014 |
| trace_acc | 0.331 | 0.241 |
| hits_celerity | 0.449 | 0.381 |
| trace_mean_err | 0.126 | 0.132 |
| trace_celerity | 0.533 | 0.390 |
| means_vel | 0.845 | 0.583 |
| trace_mean_err_rate | 0.409 | 0.210 |
| begin_trace_dist | −0.028 | 0.048 |
| end_trace_dist | 0.023 | −0.008 |
| first_last_point_dist | 0.006 | −0.009 |
| median_vel | 0.841 | 0.581 |
| cv_vel | 0.156 | 0.161 |
| cd_vel | 0.280 | 0.152 |
| kurt_vel | 0.000 | −0.005 |
| skew_vel | 0.144 | 0.139 |
| peaks_vel | 0.284 | 0.248 |
| mean_acc | 0.740 | 0.443 |
| median_acc | 0.749 | 0.446 |
| cv_acc | 0.129 | 0.119 |
| cd_acc | 0.157 | 0.089 |
| kurt_acc | 0.006 | −0.004 |
| skew_acc | 0.040 | 0.022 |
| mean_jerk | 0.572 | 0.318 |
| median_jerk | 0.611 | 0.325 |
| cv_jerk | 0.056 | 0.037 |
| cd_jerk | 0.150 | 0.084 |
| kurt_jerk | 0.025 | 0.018 |
| skew_jerk | 0.069 | 0.053 |

The handcrafted features are as follows:

| Feature name | Meaning |
|---|---|
| trace_length | The total length of the path traced by |
| | the user when completing one attempt |
| | at the test. |
| trace_duration | The total time taken for the user to |
| | complete an attempt at the test. |
| begin_trace_dist | The distance from where the user first |
| | places their finger from the target |
| | start point. |
| end_trace_dist | The distance from where the user first |
| | places their finger from the target |
| | end point. |
| first_last_point_dist | [For closed path tests] The distance |
| | between where a user first places |
| | their finger, and the point at which |
| | they lift their finger from the |
| | screen. |
| median_vel | The median velocity. |
| cv_vel | Coefficient of variation of velocity. |
| cd_vel | Coefficient of dispersion of velocity. |
| kurt_vel | The kurtosis of the velocity. |
| skew_vel | The skew of the velocity. |
| mean_acc | The mean acceleration. |
| median_acc | The median acceleration. |
| cv_acc | Coefficient of variation of acceleration. |
| cd_acc | Coefficient of dispersion of acceleration. |
| kurt_acc | The kurtosis of the acceleration. |
| skew_acc | The skew of the acceleration. |
| mean_jerk | The mean jerk. |
| median_jerk | The median jerk. |
| cv_jerk | Coefficient of variation of jerk. |
| cd_jerk | Coefficient of dispersion of jerk. |
| kurt_jerk | The kurtosis of the jerk. |
| skew_jerk | The skew of the jerk. |

Similarly, the same technique was used to train a model to predict the latent variables from the handcrafted features. The first column is the index of the latent varable, The second column is the standard deviation of each variable on a test set. If this is low it means that the latent dimension was dropped out of the model can be ignored. The third column is the R² score of the predictions with the autosklearn model.

| Latent variable | Standard Deviation | R2 |
|---|---|---|
| 0 | 0.030 | 0.020 |
| 1 | 0.039 | 0.030 |
| 2 | 0.029 | 0.049 |
| 3 | 0.883 | 0.486 |
| 4 | 0.982 | 0.325 |
| 5 | 0.025 | 0.037 |
| 6 | 0.018 | 0.063 |
| 7 | 0.812 | 0.002 |
| 8 | 0.901 | 0.469 |
| 9 | 0.383 | 0.022 |
| 10 | 0.582 | 0.194 |
| 11 | 0.457 | 0.256 |
| 12 | 0.950 | 0.530 |
| 13 | 0.015 | 0.065 |
| 14 | 0.937 | 0.689 |
| 15 | 0.776 | 0.013 |
| 16 | 0.879 | 0.264 |
| 17 | 0.033 | 0.011 |
| 18 | 0.950 | 0.402 |
| 19 | 0.697 | 0.137 |
| 20 | 0.911 | 0.210 |
| 21 | 0.764 | 0.028 |
| 22 | 0.901 | 0.005 |
| 23 | 0.814 | 0.163 |
| 24 | 0.047 | 0.011 |
| 25 | 0.026 | 0.096 |
| 26 | 0.928 | 0.568 |
| 27 | 0.965 | 0.261 |
| 28 | 0.066 | 0.005 |
| 29 | 0.015 | 0.049 |
| 30 | 0.926 | 0.743 |
| 31 | 0.900 | −0.008 |
| 32 | 1.002 | 0.478 |
| 33 | 0.022 | 0.080 |
| 34 | 0.034 | 0.012 |
| 35 | 0.910 | 0.443 |
| 36 | 0.027 | 0.037 |
| 37 | 0.713 | −0.015 |
| 38 | 0.567 | 0.001 |
| 39 | 0.915 | 0.035 |
| 40 | 0.642 | 0.227 |
| 41 | 0.779 | −0.014 |
| 42 | 0.033 | 0.019 |
| 43 | 0.021 | 0.082 |
| 44 | 0.754 | −0.014 |
| 45 | 0.015 | 0.068 |
| 46 | 0.023 | 0.050 |
| 47 | 0.848 | 0.360 |
| 48 | 0.987 | 0.505 |
| 49 | 0.027 | 0.020 |

The results show how the neural networks are encoding certain handcrafted features. This shows that the autoencoder primarily focuses on encoding information and drawing speed. When looking at the results from the autosklearn regressor it may be seen that trace accuracy is also encoded. This is probably because the errors from the ideal shape are encoded in multiple latent dimensions, which would make it difficult for the mutual information to recognize.

It may also be seen that whilst some latent dimensions are well predictable from the features others are completely ignored, which just means that some information is encoded by the autoencoder that is completely missed by the handcrafted features. This is expected as these latent dimensions are only needed for the full reconstruction of the original shape and ignored by our handcrafted features.

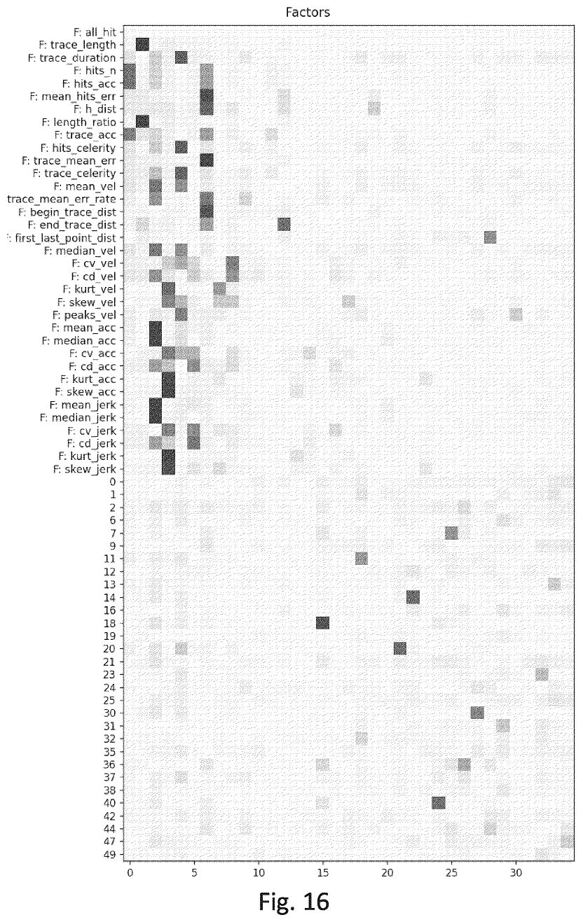

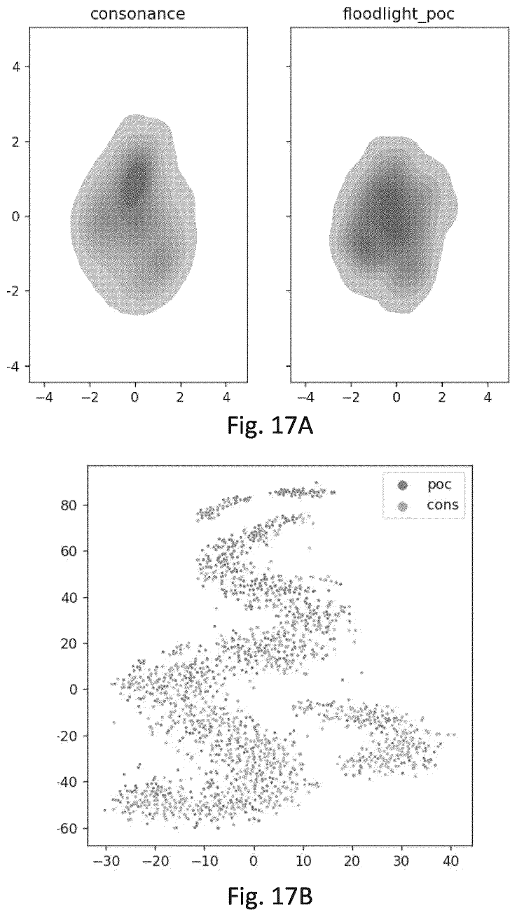

FIG. 16 shows a graph representing the result of a factor analysis, which aims to identify latent variables (“factors”) that generate observed variables. This way it is possible to see see if specific handcrafted features are encoded by the same latent factors as some of the latent dimensions. The issue here is again that this is a linear method, and the observed variables can only be a linear combination of the latent variables.

From the results it can be seen that a lot of the handcrafted features are highly correlated with each other, which is expected. As it can be seen, the individual latent dimensions are highly independent of each other, demarcated by the fact that there isn't a single component that can predict multiple latent dimensions to a high degree. Also, no single latent dimension is highly correlated with a component that is highly correlated with the handcrafted features. This is probably because the correlations are not linear and each feature is spread across multiple latent dimensions.

### Using Consonance Models on POC Data

To ascertain how transferable these models were, the models were trained on Consonance data and tested to judge their effectiveness on Floodlight POC data.

First it was tested whether using a model trained on Consonance and applying it to Floodlight POC data would still work. There did not appear to be any issue with the reconstructions. In fact, the MSE on the same validation set produces a much lower value compared to a model trained directly on POC data. This indicates that this model being trained on a larger more diverse dataset performs better.

For reference the MSE on the validation PoC data is at a KL loss weight of 1:
Model trained on PoC: 0.004
Model trained on Consonance: 0.0014

The MSE on consonance for the model trained on consonance is 0.0014, which is the same as when testing it on PoC data. This indicates that the network can apply what it has learned on consonance directly to PoC data.

It was then checked if the PCA or the t-SNE of the latent representation (from a model trained on consonance only) would show different distributions for the two datasets.

FIG. 17A is a graph of a KDE (Kernel Density Estimate) plot of the PCA of the two datasets (fitted together, then separated).

FIG. 17B is a graph of a scatterplot of the t-SNE of a sample of the POC and Consonance datasets.

The result is that they generally overlap even though they seem to have slightly different distributions.

Finally, some simple classifiers were trained to separate the datasets. This was quite successful and the two data sets can be separated reasonably well. However, some care must be taken because even just separating patients is quite easy (as per section above) so all that might be happening is training a classifier capable of identifying the style of the patients.

Validation accuracy (balanced datasets):
LogReg: 0.698
RF: 0.728
KNN: 0.742
SVM: 0.772
GNB: 0.681

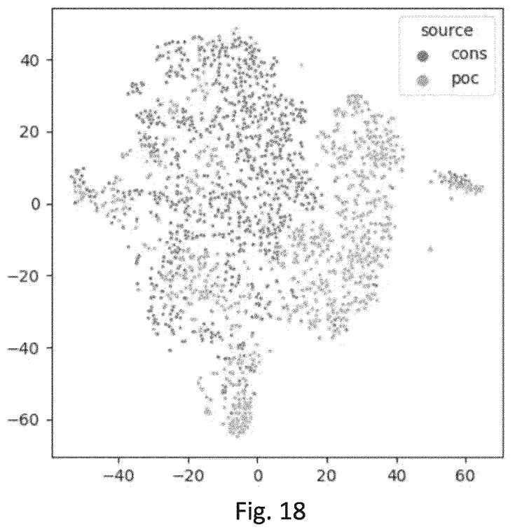

It was tested to see how different the data was when looking at the handcrafted features and we noticed that in some features the data differed substantially. This is shown in FIG. 18, which is a scatterplot of a t-SNE representation of a sample of the two datasets by handcrafted features.

### Predicting 9HPT Times

One of the objectives was also to test if the features the models capture are relevant to the clinical state of the patients. This was done in multiple ways and initially completely unsupervised neural networks were tested and then finally a fully supervised approach were tested.

To properly evaluate how the network performs compared to the traditional handcrafted features, it was necessary to establish a comparable baseline processed in the same way as what the neural networks will use. This is because the way these networks work is by looking at one shape at a time, which for the task of predicting a 9HPT time is extremely bad, since the signal is very noisy between one attempt to the next.

For this task an autosklearn model was trained that takes in input the handcrafted features and tries to predict the 9HPT score for each individual drawing. After that the predicted scores are aggregated. For POC data this is aggregated per patient because the 9HPT score is averaged. For consonance the 9HPT score is assigned to the closest date for each drawing and the predictions are aggregated per visit. For consonance generating metrics per visit was also tried, but the conclusions were the same so are not presented here.

Multiple methods were tested to predict 9HPT times from the models. Work began with latent representation of the shapes from unsupervised models, but since these did not perform very well, 9HPT heads were added to the models in an attempt to force the network to pay more attention to features important for 9HPT prediction. This was implemented as a single linear layer added on top of the sampled latent output (after KL loss).

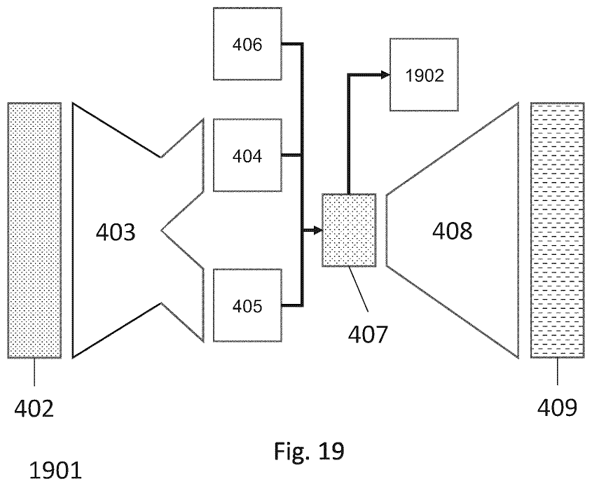

FIG. 19 shows a representation of the architecture of this modified network. This semi-supervised architecture 1901 is based off the architecture 401 represented in FIG. 4 but contains the additional feature of a 9HPT head 1902 that is derived from the bottleneck layer 407. This 9HPT head 1902 is a single linear layer. In this network the Loss=α*KL div+β*reconstruction+γ*MSE (9HPT) where α, β, and γ are weights and have been set to 1.

In the end completely supervised approaches were tested and transfer learning capabilities were tested as well. This was implemented by using the same encoder architecture but without a sampling layer or decoder. Instead, a single linear layer predicted the 9HPT value for each shape.

All the results in this section are based on the spiral shape only, but initial tests using other shapes show similar results

### Baseline Using Features

We generated a baseline using the handcrafted features to be able to compare the results generated from the neural networks. These tests were done with 5 fold cross-validation, but some results used all 5 folds whilst others only predicted 1 fold.

| Metric (CONSONANCE) | Value (1 fold of 5-fold validation) |
|---|---|
| MAE | 6.92 |
| RMSE | 10.59 |
| Pearson's correlation | 0.754 (p = 3.7e−45) |

| Metric (CONSONANCE) | Value (1 fold of 5-fold validation) |
|---|---|
| MAE | 6.92 |
| RMSE | 10.59 |
| Pearson's correlation | 0.754 (p = 3.7e−45) |
| Spearman correlation | 0.714 (p = 4.56e−38) |

| | Value (all folds of 5-fold |
|---|---|
| Metric (CONSONANCE) | validation, all samples) |
| MAE | 6.93 |
| RMSE | 10.48 |
| Pearson's correlation | 0.67 (p = 5.7e−157) |
| Spearman correlation | 0.65 (p = 6.5e−145) |

| Metric | Value (all folds of 5-fold |
|---|---|
| (FLOODLIGHT POC) | validation) |
| MAE | 2.24 |
| RMSE | 3.27 |
| Pearson's correlation | 0.68 (p = 2.9e−13) |
| Spearman correlation | 0.60 (p = 9.2e−10) |

It is interesting to also see which handcrafted features are regarded as important by the autosklearn model. Permutation importance to test which features are important. Note that if multiple features are correlated between them but they both show up, this is because autosklearn makes use of ensemble models so the importances would be distributed across them.

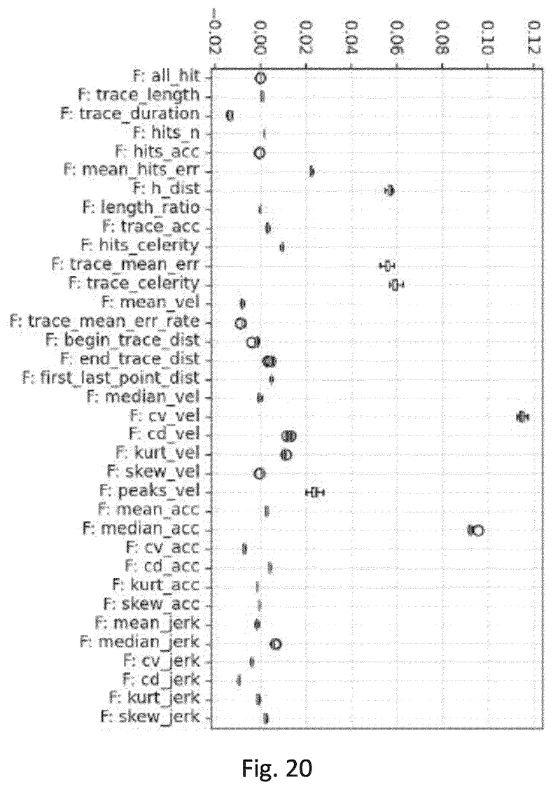

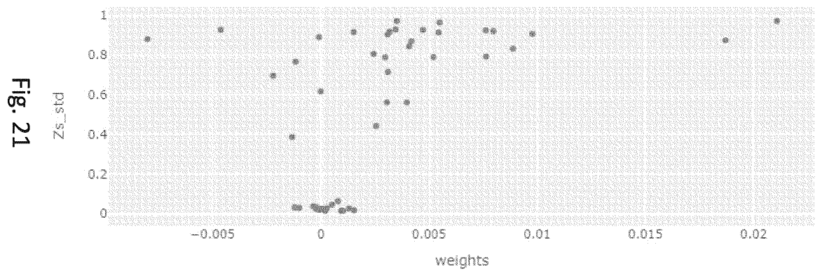

FIG. 20 shows a representation of the level of importance of each of the handcrafted features for prediction of 9HPT times.

### Using Unsupervised Models

To see if the information encoded in the latent space is useful for the prediction of 9HPT times, some models were trained that predicted 9HPT times from the latent representations of the spirals. This was tried with normal sklearn models (linear regression, decision tree regressor and gradient boosting regressor with default settings). The best result was with either the Tree regressor or the gradient boosting regressor (default hyperparameters). CNN model KL weight 1 and predictions on all 5 folds of k-fold validation:

| | | Value (all folds of 5- |
|---|---|---|
| Metric (CONSONANCE) | Baseline | fold validation) |
| MAE | 6.93 | 8.23 |
| RMSE | 10.48 | 12.32 |
| Pearson's correlation | 0.67 (p = 5.7e−157) | 0.50 (p = 6.57e−78) |
| Spearman correlation | 0.65 (p = 6.5e−145) | 0.536 (p = 2.70e−89) |

We then tried using autosklearn which resulted in significantly better predictions:

| | | Value (1 fold of 5- |
|---|---|---|
| Metric (CONSONANCE) | Baseline | fold validation) |
| MAE | 6.92 | 7.99 |
| RMSE | 10.59 | 12.32 |
| Pearson's correlation | 0.754 (p = 3.7e−45) | 0.58 (p = 7.9e−23) |
| Spearman correlation | 0.714 (p = 4.56e−38) | 0.61 (p = 1.17e−25) |

The table shows that the results are still significantly worse than using the handcrafted features. This would indicate that although some useful information is encoded, a lot of useful information is lost. Results from KL loss weight of 0.1 and 0 are very similar.

Running permutation importance on this model identified the most useful latents in predicting 9HPT times. This is shown in FIG. 21 which is a graph that represents permutation importance weights vs standard deviation of the individual latents.

From the graph, it can be seen that the model learns that latents dimensions that collapsed have very little weight on 9HPT prediction, whilst others have more importance.

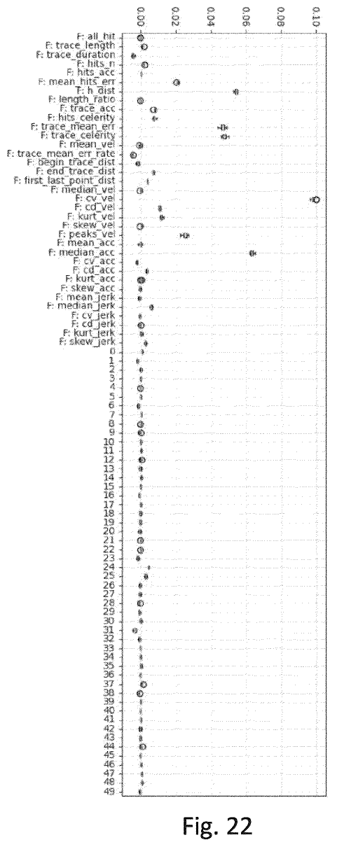

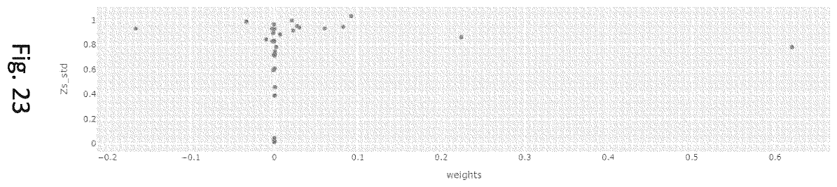

It was tested to determine if an autosklearn model trained to predict 9HPT would prefer using handcrafted features or latent dimensions. This is to see if the autoencoder is encoding different information compared to the handcrafted features, in which case the combination of the two would produce even better results. The permutation importance results shown in FIG. 22 show that the model prefers the handcrafted features over the latent representations from the unsupervised model.

Models with 9HPT Head

Next analysis was performed to determine if adding an 9HPT prediction head to the model would push the model to learn more useful features for the 9HPT prediction and also incorporate that information in the reconstructions. The idea is that because of the KL loss if something is needed for the 9HPT prediction then it should also be used by the decoder to improve the reconstruction. Those latent dimensions can then be varied to see its effects on the reconstruction. Albeit when tested, this did not produce explainable changes.

Two observations were made, first the networks tend to overfit significantly on the training set. This could be alleviated but not eliminated by using a lower weight for this output and using a MAE loss instead of a MSE loss. Regardless, the actual predictions on the validation set are pretty good (better than baseline).

| | | Value (1 fold of 5- |
|---|---|---|
| Metric (CONSONANCE) | Baseline | fold validation) |
| MAE | 6.92 | 6.51 |
| RMSE | 10.59 | 9.78 |
| Pearson' s correlation | 0.754 (p = 3.7e−45) | 0.68 (p = 7.1e−35) |
| Spearman correlation | 0.714 (p = 4.56e−38) | 0.58 (p = 6.2e−24) |

| | | Value (all folds of 5- |
|---|---|---|
| Metric (CONSONANCE) | Baseline | fold validation) |
| MAE | 6.93 | 6.55 |
| RMSE | 10.48 | 10.46 |
| Pearson' s correlation | 0.67 (p = 5.7e−157) | 0.71 (p = 4e−189) |
| Spearman correlation | 0.65 (p = 6.5e−145) | 0.67 (p = 1.7e−154) |

| Metric (FLOODLIGHT | | Value (all folds of 5- |
|---|---|---|
| POC) | Baseline | fold validation) |
| MAE | 2.24 | 2.26 |
| RMSE | 3.27 | 3.43 |
| Pearson' s correlation | 0.68 (p = 2.9e−13) | 0.61 (p = 3.2e−10) |
| Spearman correlation | 0.60 (p = 9.2e−10) | 0.55 (p = 5.2e−08) |

### Searching for Encoded Features

Next was to identify which features encoded in the latent dimensions were useful for 9HPT reconstruction. Because this prediction is implemented as a single linear layer it is possible to check the weights of this layer, the higher the weight the more important that latent dimensions is.

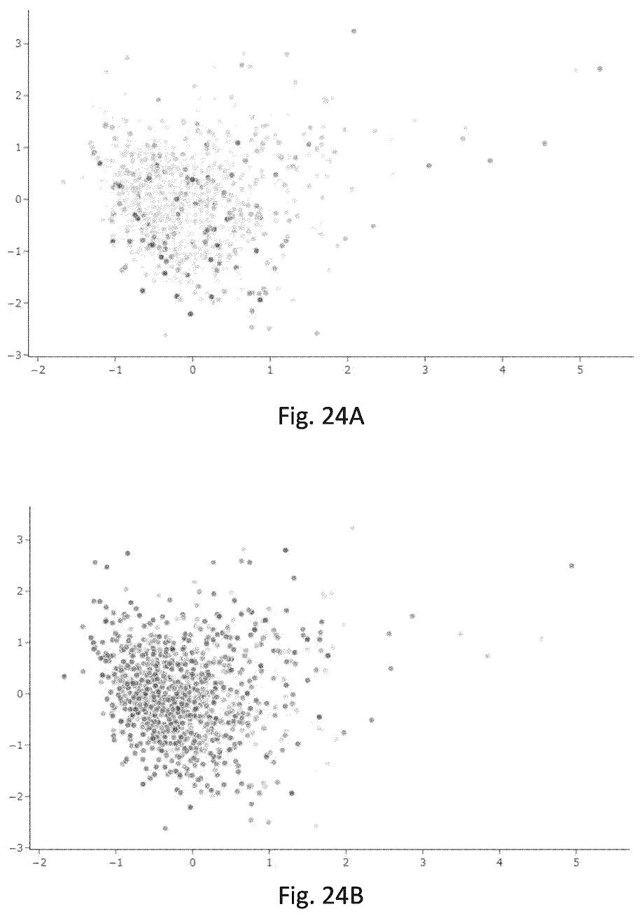

FIG. 23 shows the layer weights vs the standard deviation of the latent dimensions. In this model the top 3 latents were 44, 7, and 40, in this instance.

A typical situation is that one latent contributes most of the score, with another 1 or 2 latent dimensions giving a much lower weight and the rest do not really contribute to the final score.

Interestingly if the data is plotted using the two top latent dimensions as X and Y dimensions the data seems to separate quite well by trace celerity, but with 9HPT the separation is not as stark. This is shown in FIGS. 24A and 24B, where the axis are the two top latent dimensions, and in 24A the points are coloured by trace celerity, and in 24B the points are coloured by 9HPT times.

The techniques used with fully unsupervised models were then applied to the 9HPT models to identify encoded features using mutual information and autosklearn. The results show that the latent dimensions used for 9HPT prediction do not seem to encode any information from the handcrafted features. Moreover, the model trained to predict the latents from the features is not able to predict those 3 latents.

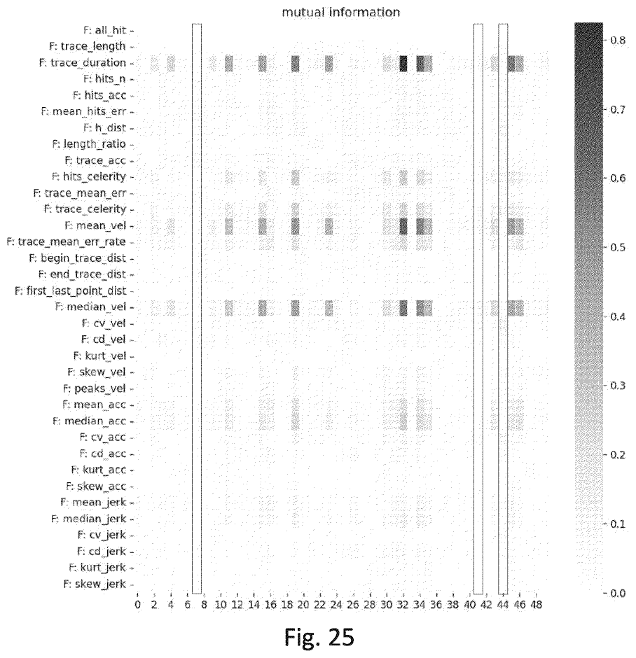

This can be seen from the heatmap shown in FIG. 25. The heatmap shows the amount of mutual information between every latent dimension and handcrafted feature. Notably, dimensions 7, 40, and 44 (boxed) seem not to encode any features.

Given this change, analysis was performed to determine the model with both handcrafted features and latent representations would prefer to use the latent representations or the handcrafted features.

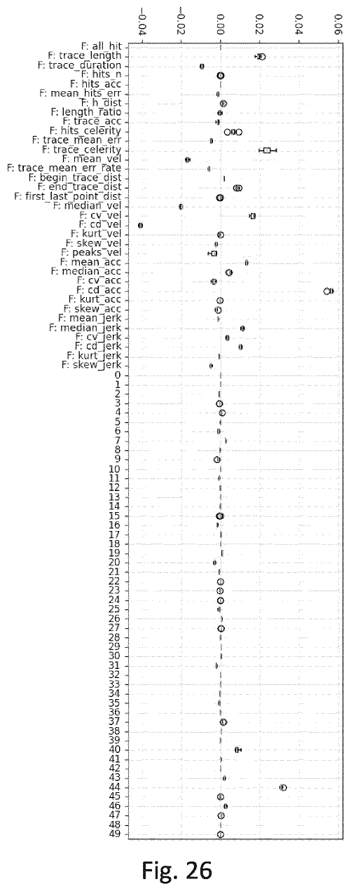

For this the autosklearn model was trained on a subset of the validation set, so that the model would not be trained on an overfitted dataset. Nevertheless it can be seen in FIG. 26 that the permutation importance shows that the top dimensions used by the network (44, 40 and 7) are important in the prediction of 9HPT times. This indicates that the network is actually providing useful information to the model even if it is overfitting the training set.

The models trained here can predict 9HPT times, possibly better than using the handcrafted features. How this prediction is made is not certain but it may be because the last layer is only a single linear layer, the actual features are located upstream of the sampling layer and in the sampling layer only a single or a few dimensions are passed on as the prediction for 9HPT times.

### Completely Supervised Models

Next a completely supervised model trained in the same way was tested. This model does not have a KL loss and a decoder section, but for the rest of the supervised model is the same as the previous encoder models.

The results are very similar to the model with a decoder, meaning that both requiring the reconstruction and the sampling process do not impede the quality of the prediction.

| | | Value (all folds of 5- |
|---|---|---|
| Metric (CONSONANCE) | Baseline | fold validation) |
| MAE | 6.93 | 6.51 |
| RMSE | 10.48 | 10.29 |
| Pearson' s correlation | 0.67 (p = 5.7e−157) | 0.68 (p = 3.6e−164) |
| Spearman correlation | 0.65 (p = 6.5e−145) | 0.66 (p = 9.9e−151) |

| Metric (FLOODLIGHT | | Value (all folds of 5- |
|---|---|---|
| POC) | baseline | fold validation) |
| MAE | 2.24 | 2.23 |
| RMSE | 3.27 | 3.39 |
| Pearson's correlation | 0.68 (p = 2.9e−13) | 0.64 (p = 3.68e−11) |
| Spearman correlation | 0.60 (p = 9.2e−10) | 0.61 (p = 6.8e−10) |

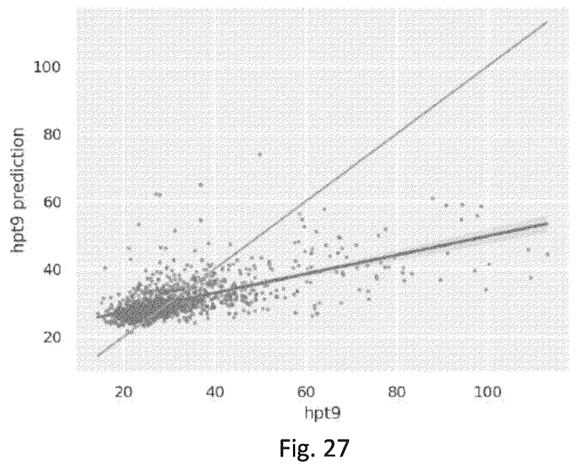

FIG. 27 shows a scatter plot of Consonance 9HPT times prediction per patient visit from fully supervised models with a blue trend line, overlayed with an idealised red line where prediction matches the true time.

### Transfer Learning

Transfer learning was explored to determine its usefulness, by extracting the encoder sections from the autoencoder models and using them for 9HPT prediction. The Consonance dataset is many times larger than the Floodlight POC dataset. The same testing was performed for the autoencoder models with the 9HPT head but the results are the same so detailed results are not presented here.

The following combinations were tested:
Directly using a consonance model with no finetuning at all on floodlight poc
Pretrained fully supervised model then fine tuned (trained without decoder)
Pretrained unsupervised model then fine tuned (trained autoencoder without 9HPT head)
Pretrained supervised model then fine tuned (trained autoencoder with 9HPT head)

Compared to the baseline the models trained with a supervised approach can significantly improve the result compared to a model that was trained only on Floodlight data, whilst the model trained in an unsupervised way did not improve the result. It is possible that the network will only learn features useful for 9HPT prediction if it is directed to do so and the pure autoencoder models fail to capture this information. It can be seen that a Consonance model not fine tuned at all still works.

A pretrained unsupervised model was also tested on Consonance data that is trained on the full dataset including unlabelled samples, but this pre-training actually performs worse than no pre-training at all, reinforcing the previous conclusion that unsupervised models capture different features than the ones useful for 9HPT prediction.

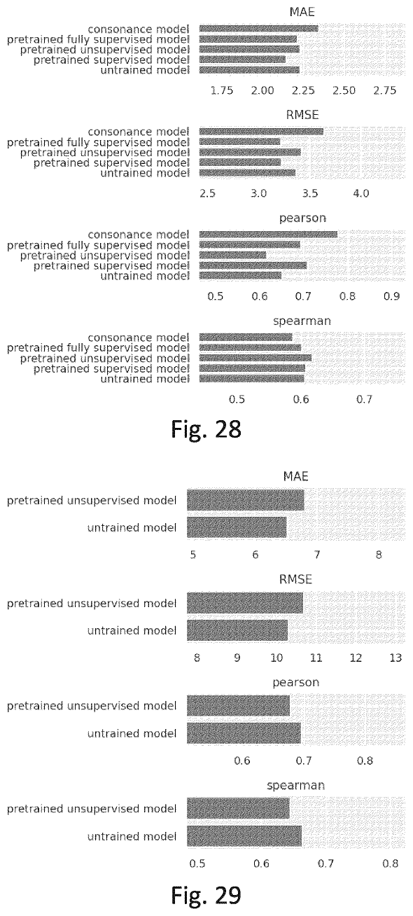

The result for this can be seen in the graphs on FIGS. 28 and 29. FIG. 28 shows the results for these transfer learning experiments on Floodlight POC data. FIG. 29 shows the results for these transfer learning experiments on Consonance data.

### Predicting 9HPT Over Time

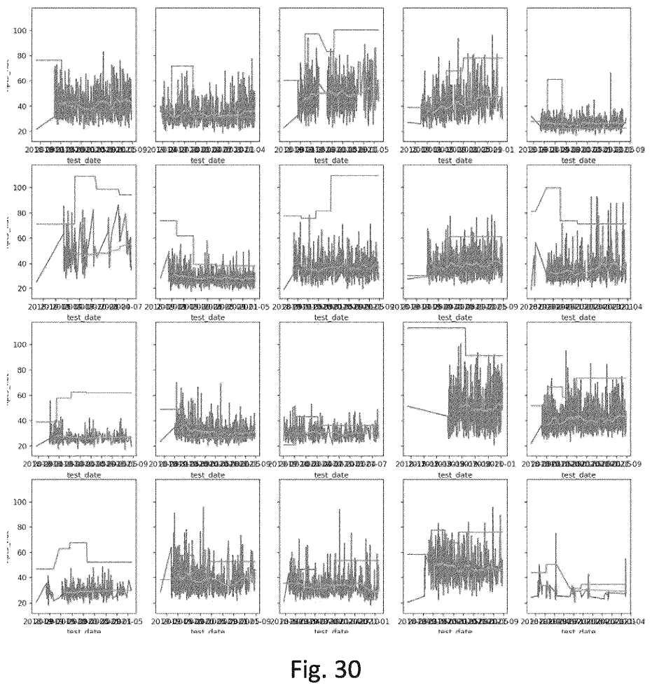

The models were tested to see if they could identify significant longitudinal differences in Consonance patients that have significant clinical progression. There does not seem to any obvious correlation when looking at individual patients, nor when grouping predictions per visit. FIG. 30 displays an array of graphs each representing a selection of the data from Consonance patients with large 9HPT test time changes. The green line is the clinical 9HPT times, the blue line is the prediction from the fully supervised models, and in orange a moving average of these predictions.

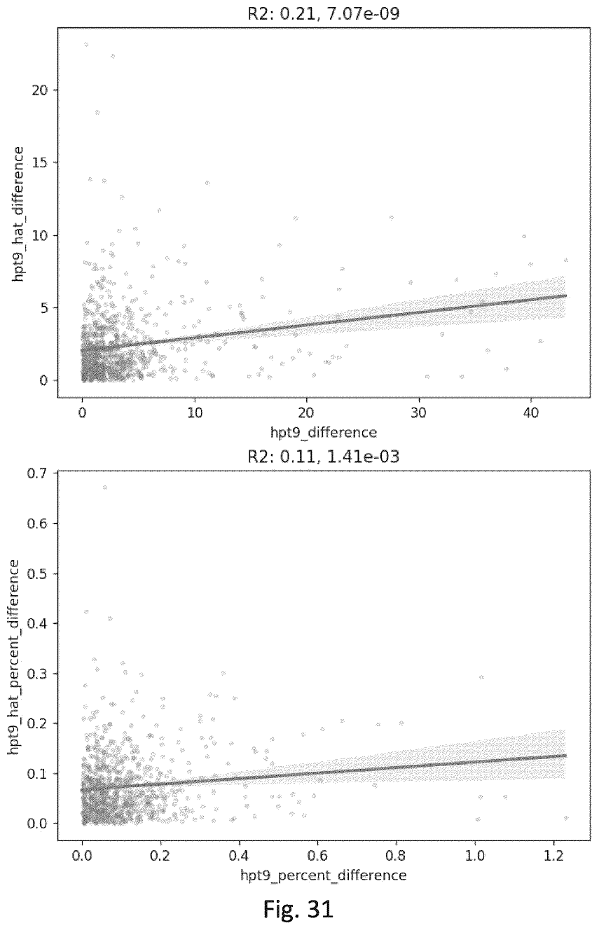

FIG. 31 shows two scatterplot graphs of real 9HPT differences vs predicted 9HPT differences in Consonance patients. This takes the drawings made within 1 month of visit dates and aggregates the predictions per visit, then calculates the difference from baseline (first visit), in absolute differences (top graph) and percent differences (bottom graph).

### CONCLUSION

From these results it can be concluded that:
Neural networks are able to encode more useful information for the prediction of 9HPT times than the handcrafted features since the predictions from supervised models are more accurate.
Pretraining and fine-tuning helps if the networks were pre-trained in a supervised setting
Training the network with all shapes gives similar results

### Progression Analysis

Since the consonance study contains data over a long time period, it was explored if it was possible to detect changes in the drawing style of patients.

This task is rendered very complicated because the CNN models produce a very high dimensionality space. Initially Euclidean distances were calculated based on the latent space of these models, but the results were difficult to interpret because all shapes seemed to be about the same distance from each other, probably because of the high dimensionality.

For this reason, UMAP transformation was used as described in McInnes, L, Healy, J, UMAP: Uniform Manifold Approximation and Projection for Dimension Reduction, ArXiv e-prints 1802.03426, 2018. However distances calculated on UMAP also have no significance, but at least it is possible to visualize if changes are present.

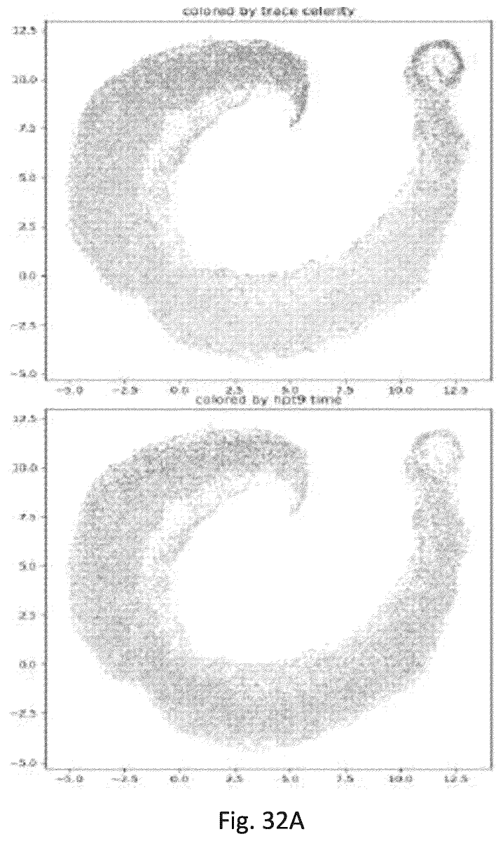

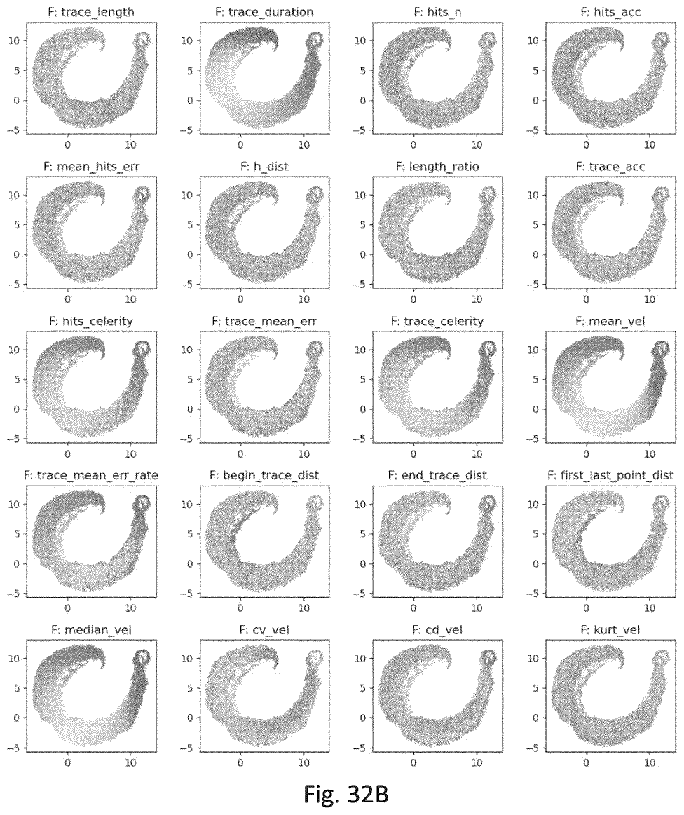

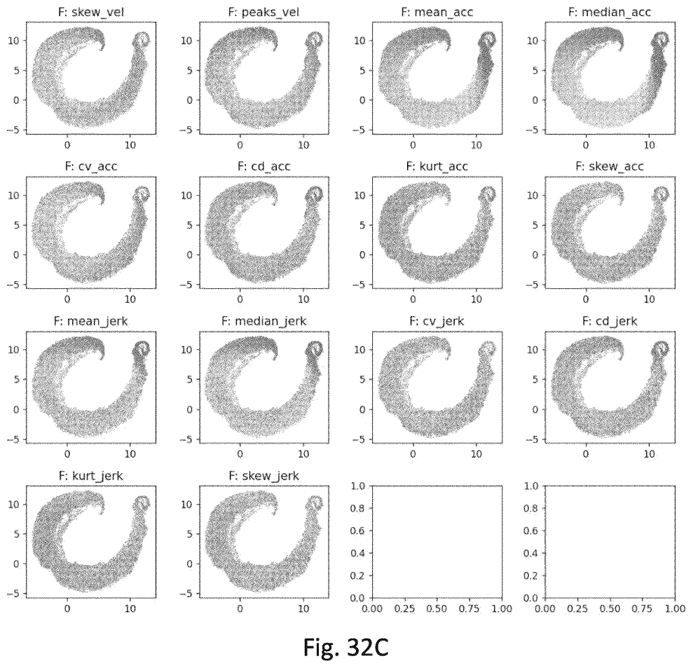

One way to visualize drawing style change is to see if the latent representations of their drawings move around over time. The UMAP algorithm was run on a subset of 50 random patients creating the projection shown in FIG. 32A. In this graph, the top plot is coloured by trace celerity, and the bottom plot is coloured by 9HPT time.

FIGS. 32B and 32C each show a series of graphs for the same UMAP, each coloured for a different handcrafted feature. From the graphs, it can be seen that the main differentiating factor in the space is the trace duration (which correlated with celerity). On a secondary axis, the data is still separated by some of the other handcrafted features.

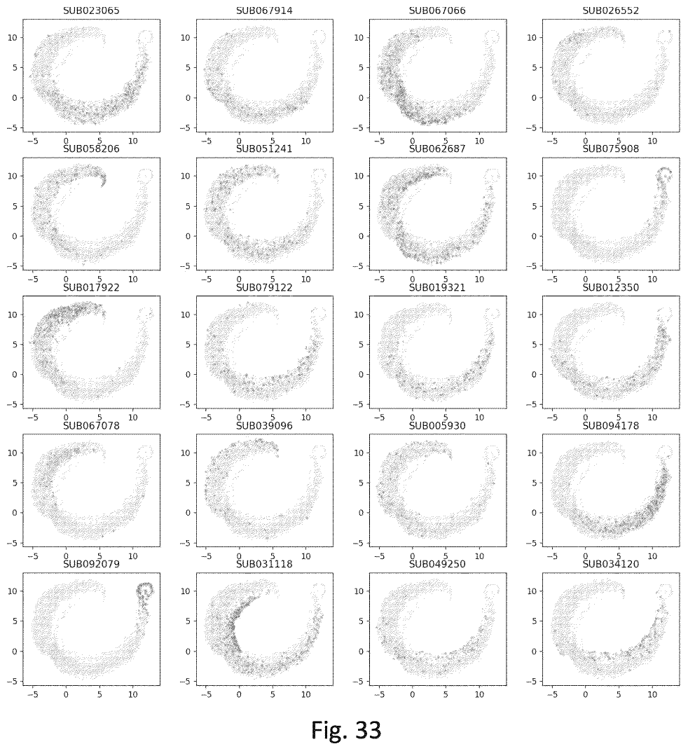

FIG. 33 shows a series of graphs each containing the UMAP of the latent space of the CNN model, wherein each graph highlights the data points for a single patient. These data points are coloured based on the test date on which the data was obtained with blue representing earlier points and red later points. From these graphs, it can be seen that for some patients the position of the drawings in the latent spaces changes, indicating that the drawing style of each patient changes over time.

The most interesting patients are the ones that start in some region of the latent space and then move around. These are the patients for which the drawing style significantly changes over time. Specifically, since the network seems to place a high significance on drawing velocity, the separation is most evident in patients that have increased or decreased their drawing speed over time. Other features are also encoded, for example subject 062687 (third column second row) moves in a region with lower cv acc and higher cv_vel and cd_vel.

Subject 031118 (second column fifth row) shows another interesting phenomena, in which his later drawings are all located in this separate island. Visual inspection of those drawings didn't show any specific difference but colouring the latent space with the other handcrafted features shows that the region has some specific characteristics (with h_dist, first_last_point_dist and begin_trace_dist).

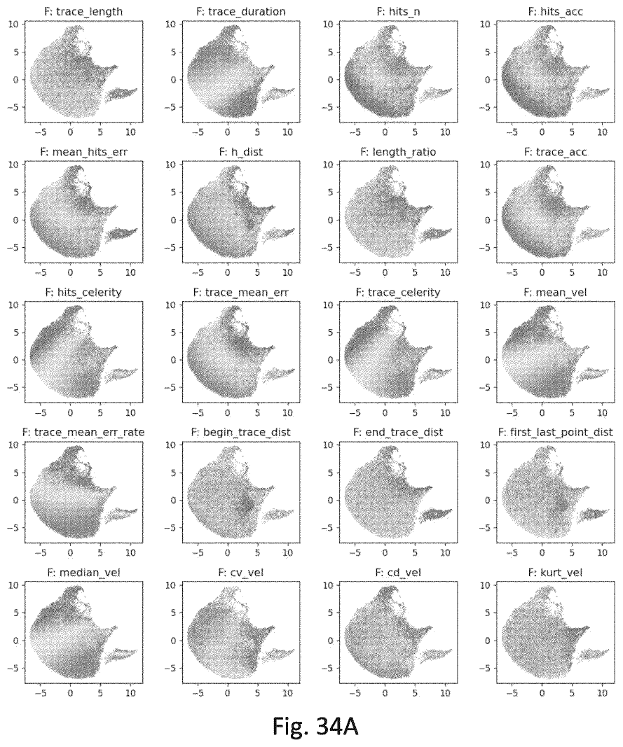

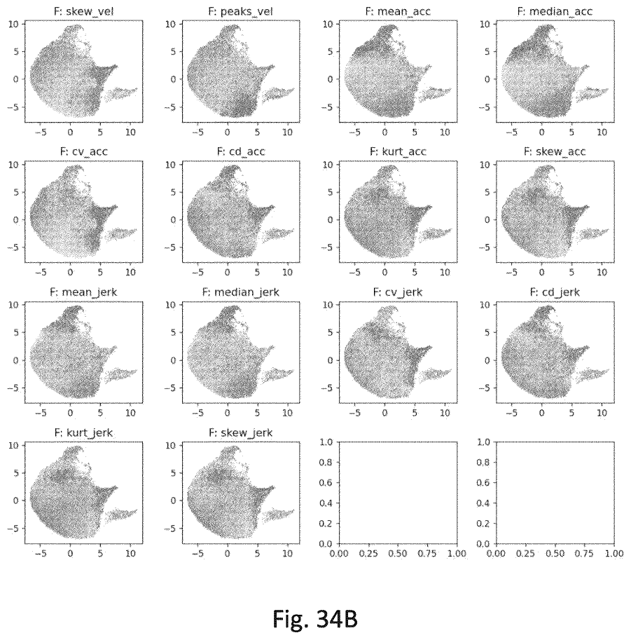

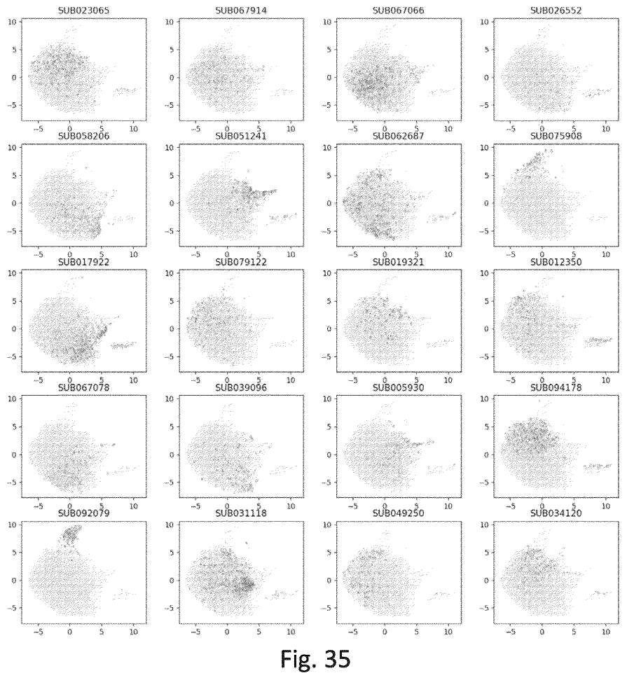

One argument about what we have just done though is how much of what we see is already incorporated into the handcrafted features. To verify this we ran a UMAP using the handcrafted features and in fact we can see how patients move around in the UMAP space in a similar way. FIGS. 34A and 34B show these UMAPs coloured by each handcrafted feature. Features were standardised before manifold discovery. FIG. 35 shows the same UMAPs highlighting the results from individual patients coloured by test time.

It is possible that the neural network would have separated the data in a different way since from previous sections we know that some latent variables are not encodable by the handcrafted features, but at this point this has not yet been verified.

The following numbered clauses contain statements of broad combinations of the inventive technical features herein disclosed:

1. A computer-implemented method of generating an analytical model for tracking or predicting the progression of a neurological impairment, the computer-implemented method comprising:
receiving training data comprising the results of a plurality of digital tests of neurological impairment; and
training the analytical model using the received training data, thereby generating the analytical model.

2. A computer-implemented method according to clause 1, wherein:
the analytical model is a machine-learning model comprising an encoder configured to generate, from an input data set comprising a first number of variables, a latent representation of the input data set comprising a second number of latent variables, the second number being less than the first number.

3. A computer-implemented method according to clause 2, wherein:
the training data comprises a plurality of input data sets each comprising a first number of variables; and
training the analytical model comprises training the encoder to learn a respective latent representation of the plurality of input data sets of the training data, wherein each respective latent representation comprises a second number of latent variables, the second number being less than the first number.

4. A computer-implemented method according to clause 2 or clause 3, wherein:
the machine-learning model is a variational autoencoder comprising the encoder; and
the encoder has been trained or is trained in an unsupervised manner as part of the variational autoencoder.

5. A computer-implemented method according to clause 4, wherein:
the encoder comprises a latent distribution determination module configured to determine, for each of the latent variables, a respective latent distribution;
each latent distribution is a probability distribution for the value of the latent variable corresponding to the respective dimension in the latent space.

6. A computer-implemented method according to clause 4 or clause 5, wherein the variational autoencoder further comprises a decoder configured to:
generate, from the latent representation comprising the second number of latent variables, an output data set comprising a third number of variables, the third number being greater than the second number; or
generate, from an input data set comprising the latent variables of the encoder, an output data set that reproduces the input data provided to the encoder.

7. A computer-implemented method according to any one of clauses 1 to 6, further comprising:
at least partially retraining the encoder previously trained using different training data;
and/or wherein training the encoder is performed by transfer learning.

8. A computer-implemented method according to any one of clauses 1 to 7, wherein:
training the encoder comprises training the encoder as part of an analytical model configured to predict one or more metrics indicative of the status or progression of neurological impairment; and/or
training the encoder model comprises training the encoder model in a supervised manner using training data comprising the value of one or more metrics indicative of the status or progression of neurological impairment.

9. A computer-implemented method according to any one of clauses 1 to 8, wherein:
the data comprising the results of the digital test of neurological impairment comprises a plurality of coordinates, each coordinate corresponding to a location of a user's finger on the touchscreen display of an electronic device at a given time, as they attempt to trace the target shape.

10. A computer-implemented method according to any one of clauses 1 to 9, wherein:
the neurological impairment is multiple sclerosis.

11. A computer-implemented method of extracting feature data from results of a digital test of neurological impairment, the computer-implemented method comprising:
receiving data comprising the results of a digital test of neurological impairment;
applying an analytical model to the data, the analytical model configured to extract and output the feature data based on the data comprising the results of the digital test of neurological impairment, wherein the analytical model is generated according to the computer-implemented method of any one of clauses 1 to 10.

12. A computer-implemented method according to clause 11, wherein:
the analytical model comprises an encoder configured to generate, from an input data set comprising a first number of variables, a latent representation of the input data comprising a second number of latent variables, the second number being less than the first number; and
the computer-implemented method further comprises extracting the feature data from the latent representation of the input data.

13. A computer-implemented method of tracking or predicting the progression of a neurological impairment or other disease in a subject, the computer-implemented method comprising the steps of:
extracting feature data from results of a digital test of neurological impairment performed by the subject according to the computer-implemented method of any one of clauses 1 to 12; and
determining or predicting the status or progression of the neurological impairment based on the extracted feature data.

14. A computer-implemented method according to clause 13, wherein:
determining the status or progression of the neurological impairment based on the extracted feature data comprises comparing the value of one or more latent variables from the latent representation of the data with one or more reference values.

15. A computer-implemented method according to clause 14, wherein the reference values are values of the latent variables obtained for one or more reference results of a digital test of neurological impairment.

1. A computer-implemented method of tracking or predicting the progression of a neurological impairment or other disease in a subject, the computer-implemented method comprising the steps of:
extracting feature data from results of a digital test of neurological impairment performed by the subject by using an analytical model comprising an encoder configured to generate a latent representation comprising one or more latent variables; and
determining or predicting the status or progression of the neurological impairment based on the extracted feature data by comparing the value of the one or more latent variables with one or more reference values
wherein the results of the digital test of neurological impairment comprises a plurality of coordinates, each coordinate corresponding to a location of a user's finger on the touchscreen display of an electronic device at a given time, as they attempt to trace a target shape.

2. A computer-implemented method according to claim 1, wherein the reference values are values of the latent variables obtained for one or more reference results of a digital test of neurological impairment.

3. A computer-implemented method of generating an analytical model for tracking or predicting the progression of a neurological impairment, the computer-implemented method comprising:
receiving training data comprising the results of a plurality of digital tests of neurological impairment; and
training the analytical model using the received training data, thereby generating the analytical model,
wherein the training data comprising the results of the digital test of neurological impairment comprises a plurality of coordinates, each coordinate corresponding to a location of a user's finger on the touchscreen display of an electronic device at a given time, as they attempt to trace a target shape.

4. A computer-implemented method according to claim 3, wherein:
the analytical model is a machine-learning model comprising an encoder configured to generate, from an input data set comprising a first number of variables, a latent representation of the input data set comprising a second number of latent variables, the second number being less than the first number.

5. A computer-implemented method according to claim 4, wherein:
the training data comprises a plurality of input data sets each comprising a first number of variables; and
training the analytical model comprises training the encoder to learn a respective latent representation of the plurality of input data sets of the training data, wherein each respective latent representation comprises a second number of latent variables, the second number being less than the first number.

6. A computer-implemented method according to claim 4 or claim 5, wherein:
the machine-learning model is a variational autoencoder comprising the encoder; and
the encoder has been trained or is trained in an unsupervised manner as part of the variational autoencoder.

7. A computer-implemented method according to claim 6, wherein:
the encoder comprises a latent distribution determination module configured to determine, for each of the latent variables, a respective latent distribution;
each latent distribution is a probability distribution for the value of the latent variable corresponding to the respective dimension in the latent space.

8. A computer-implemented method according to claim 6 or claim 7, wherein the variational autoencoder further comprises a decoder configured to:
generate, from the latent representation comprising the second number of latent variables, an output data set comprising a third number of variables, the third number being greater than the second number; or
generate, from an input data set comprising the latent variables of the encoder, an output data set that reproduces the input data provided to the encoder.

9. A computer-implemented method according to any one of claims 3 to 8, further comprising:
at least partially retraining the encoder previously trained using different training data;
and/or wherein training the encoder is performed by transfer learning.

10. A computer-implemented method according to any one of claims 3 to 9, wherein:
training the encoder comprises training the encoder as part of an analytical model configured to predict one or more metrics indicative of the status or progression of neurological impairment; and/or
training the encoder model comprises training the encoder model in a supervised manner using training data comprising the value of one or more metrics indicative of the status or progression of neurological impairment.

11. A computer-implemented method according to any one of claims 1 to 10, wherein:
the neurological impairment is multiple sclerosis.
